(19) United States
(12) Patent Application Publication
(10) Pub. No.: US 2025/0295759 A1
(43) Pub. Date: Sep. 25, 2025

(54) METHOD FOR ENHANCING IMMUNITY

(71) Applicants: Xanadu Bio, Inc., Gladwyne, PA (US); YALE UNIVERSITY, New Haven, CT (US)

(72) Inventors: Akiko IWASAKI, Gladwyne, PA (US); W. Mark SALTZMAN, Gladwyne, PA (US); Benjamin GOLMAN-ISRAELOW, Gladwyne, PA (US); Tianyang MAO, Gladwyne, PA (US); Bruce TURNER, Gladwyne, PA (US); Todd WIDER, Gladwyne, PA (US)

(21) Appl. No.: 18/861,366

(22) PCT Filed: Apr. 28, 2023
(86) PCT No.: PCT/US2023/066330

§ 371 Date: Oct. 29, 2024

Related U.S. Application Data
(60) Provisional application No. 63/336,499, filed on Apr. 29, 2022.
(60) Provisional application No. 63/348,451, filed on Jun. 2, 2022.

(51) Int. Cl. A61K 39/215; A61K 39/00; A61K 39/12; A61K 39/385; A61P 37/04

(52) U.S. Cl. CPC A61K 39/215; A61K 39/12; A61K 39/385; A61P 37/04; A61K 2039/53; A61K 2039/541; A61K 2039/542; A61K 2039/543; A61K 2039/545; A61K 2039/575

(57) ABSTRACT

The invention relates to a method of enhancing immunity, mRNA-based vaccines for SARS-COV-2 have demonstrated the enormous potential of mRNA therapeutics for safe and effective use in the general population. However, more recent studies have demonstrated decreasing vaccine effectiveness in terms of asymptomatic infection as well as symptomatic and severe infections starting around 4 months post second dose with mRNA-lipid nanoparticles (LNP) based regimens.

### CROSS-REFERENCE TO RELATED APPLICATIONS

This application claims the benefit of U.S. Provisional Patent Application No. 63/336,499 filed Apr. 29, 2022 entitled “METHOD FOR ENHANCING IMMUNITY”, and U.S. Provisional Patent Application No. 63/648,451 filed Jun. 2, 2022 entitled “METHOD FOR ENHANCING IMMUNITY”, each of which is incorporated by reference herein in its entirety.

### FIELD OF THE INVENTION

This invention relates to a method to enhance immunity.

### SEQUENCE LISTING

The instant application contains a Sequence Listing that has been submitted electronically in XML file format and is hereby incorporated by reference in its entirety. The Sequence Listing for this application is labeled “130481-5002-WO.XML”, which was created on Apr. 25, 2023, and is 8,500 bytes in size.

### BACKGROUND OF THE INVENTION

mRNA-based vaccines for SARS-COV-2 have demonstrated the enormous potential of mRNA therapeutics for safe and effective use in the general population. However, more recent studies have demonstrated decreasing vaccine effectiveness in terms of asymptomatic infection as well as symptomatic and severe infections starting around 4 months post second dose with mRNA-lipid nanoparticles (LNP) based regimens. Furthermore, continued viral evolution with increasing immune evasiveness notably with Beta (B.1.351), Delta (B.1.617.2), and now Omicron (B.1.529) variants of concern (VOC), has also contributed to decreased vaccine effectiveness against COVID-19. Not only have current vaccines become less effective at preventing SARS-COV-2 infection, but they have also become less able to prevent viral transmission.

Therefore, there remains a need for enhancing immunity against COVID-19. The present invention meets such need.

### BRIEF DESCRIPTION OF THE DRAWINGS

FIG. 1A illustrates an experiment in which K18-hACE2 mice were intramuscularly (IM) immunized with 1 μg of mRNA-lipid nanoparticles (LNP) encoding full-length SARS-CoV-2 (SCV2) spike protein, followed by intranasal (IN) immunization with 1 μg of prefusion-stabilized, trimeric, recombinant SARS-COV-2 (SCV2) spike protein 14 days following mRNA-LNP immunization. Fourteen days post IN boost, serum, bronchoalveolar lavage fluids (BALF), and nasal washes were collected to assess binding and neutralizing antibody responses. Lung tissues were collected for extravascular B cell analysis.

FIG. 1B shows the measurement of SCV2 spike S1 subunit-specific nasal wash IgA (B), nasal wash IgG (C), BALF IgA (D), BALF IgG (E), serum IgA (F), and serum Ig (G) in naïve mice, mice immunized with mRNA-LNP IM (IM Prime), mice immunized with the spike protein IN (IN Spike), or mice IM primed and IN boosted with spike (Prime and Spike).

FIG. 1C shows the measurement of neutralization titer against SCV2 spike-pseudotyped vesicular stomatitis virus (VSV) in BALF (H,I) and serum (J,K).

FIG. 1D shows the measurement of various extravascular (intravenous labeling antibody negative) B cell subsets, including RBD tetramer-binding B cells, IgA⁺ resident memory B cells (B_{RM}), IgG⁺ B_{RM}, IgA⁺ antibody secreting cells (ASC), and IgG⁺ ASC in lung tissues from IM Prime or Prime and Spike mice.

FIG. 2A illustrates an experiment in which K18-hACE2 mice were IM primed with 1 μg mRNA-LNP and 14 days later IN boosted with 1 μg SCV2 spike. Lung tissues, BALF, and nasal turbinates were collected for extravascular T cell analysis. Lung tissues were collected 14 days post boost, BALF and nasal turbinates 7 days post boost.

FIG. 2B shows extravascular CD8 T cell responses: Quantification of SCV2 spike-specific Tetramer⁺ CD8 T cells, CD69 CD103⁻Tetramer⁺ CD8 T cells, or CD69⁺CD103⁺Tetramer⁺ CD8 T cells in lung tissues (B-D), BALF (E-G).

FIG. 2C shows extravascular CD8 T cell responses: Quantification of SCV2 spike-specific Tetramer⁺ CD8 T cells, CD69⁺CD103⁻Tetramer⁺ CD8 T cells, or CD69⁺CD103⁺Tetramer⁺ CD8 T cells in nasal turbinates (H-J), (K-P) Extravascular CD4 T cell responses: Quantification of activated polyclonal CD4 T cells, CD69⁺CD103⁻CD4 T cells, or CD69⁺CD103⁺ CD4 T cells in lung tissues (K-M) or BALF (N-P) from naïve, IM Prime, IN Spike, or Prime and Spike mice.

FIG. 3A illustrates an experiment in which K18-hACE2 mice were IM primed with 1 μg of mRNA-LNP, followed by IN boosting with 1 μg of naked mRNA (IN naked mRNA) or 1 μg of mRNA encapsulated by PACE (IN PACE-Spike) 14 days post IM Prime. Fourteen (14) days post IN boost, BALF and blood were collected for antibody measurement. Lung tissues were collected for CD8 T cell analysis.

FIG. 3B shows quantification of total Tetramer⁺ CD8 T cells, CD69⁺CD103⁻ Tetramer⁺ CD8 T cells, or CD69⁺CD103⁺Tetramer⁺ CD8 T cells in lung tissues from naïve, IM Prime, IN PACE-Spike, IM Prime+IN naked mRNA, or Prime and PACE-Spike mice.

FIG. 3C shows the measurement of SARS-CoV-2 spike S1 subunit-specific BALF IgA (E), BALF IgG (F), serum IgA (G), and serum IgG (H) in naïve, IM Prime, IN PACE-Spike, IM Prime+IN naked mRNA, or Prime and PACE-Spike mice.

FIG. 4A illustrates an experiment in which K18-hACE2 mice were IM primed with 0.05 μg of mRNALNP and IN boosted with 1 μg of spike IN 14 days post IM Prime. 6 weeks post boost, mice were challenged with 6×10⁴ PFU SARS-CoV-2 (2019n-CoV/USA_WA1/2020). The first cohort was used to evaluate weight loss and survival up to 14 days post infection (DPI). The second cohort was used to collect lung and nasal turbinate tissues 2 DPI for viral titer measurement. The third cohort was used to collect lung tissues 5 DPI for histological assessment.

FIG. 4B shows the weight loss and survival of naïve, IM Prime, or Prime and Spike mice from 1 to 14 DPI. (E-F) Measurement of infectious virus titer in lung and nasal turbinate tissues at 2 DPI by plaque assay. (G) Pathology score of lung sections at 5 DPI by Hematoxylin and Eosin (H&E) staining.

FIG. 4C shows representative H&E staining results from uninfected, IM Prime, or Prime and Spike mice.

FIG. 4D illustrates an experiment in which K18-hACE2 mice were IM primed with 0.05 μg of mRNA-LNP and IN boosted with 10 μg of mRNA encapsulated by PACE (IN PACE-Spike) 14 days post IM Prime. 6 weeks post boost, mice were challenged with 6×10⁴ PFU SARS-CoV-2 (2019n-CoV/USA_WA1/2020).

FIG. 4E shows weight loss and survival of naïve, IM Prime, or Prime and PACE-Spike K18-hACE2 mice from 1 to 14 DPI.

FIG. 5A illustrates an experiment in which K18-hACE2 mice were IM primed with 1 μg of mRNA-LNP, followed by boosting with 1 μg of mRNALNP IM, or 5 μg of prefusion-stabilized, trimeric, recombinant SARS-CoV-1 (SCV1) spike IN (IN SpikeX) 14 days post IM Prime. Thirty-one days post boost, lung tissues were collected for T cell analysis by flow cytometry, and BALF and blood were collected for antibody measurement.

FIG. 5B shows the quantification of total Tetramer⁺ CD8 T cells, CD69⁺CD103⁻ Tetramer⁺ CD8 T cells, or CD69⁺CD103⁺Tetramer⁺ CD8 T cells in lung tissues from naïve, mRNA-LNP Prime/Boost, or Prime and SpikeX mice.

FIG. 5C shows the measurement of SCV1 spike S1 subunit-specific BALF IgA (E), BALF IgG (F), serum IgA (G), and serum IgG (H) in naïve, mRNA-LNP Prime/Boost, or Prime and SpikeX mice. (I-L) Measurement of SCV2 spike S1 subunit-specific BALF IgA (I), BALF IgG (J), serum IgA (K), and serum IgG (L) in naïve, mRNA-LNP Prime/Boost, or Prime and SpikeX mice. (M,N) Measurement of neutralization titer against SCV1 spike-pseudotyped VSV. (O,P) Measurement of neutralization titer against SCV2 spike-pseudotyped VSV.

FIG. 6A illustrates an experiment in which K18-hACE2 mice were IM primed with 1 μg of mRNA-LNP and IN boosted with 1 μg SCV2 spike 12 weeks post IM Prime. Seven and 56 days post boost, lung tissues were collected for T cell analysis by flow cytometry, and BALF and blood were collected for antibody measurement.

FIG. 6B shows the (B-D) Quantification of total Tetramer⁺ CD8 T cells, CD69⁺CD103⁻ Tetramer⁺ CD8 T cells, or CD69⁺CD103⁺Tetramer⁺ CD8 T cells in lung tissues from IM Prime or Prime and Spike mice 7 and 56 days post boost. (E-G) Quantification of total activated, polyclonal CD4 T cells, CD69⁺CD103⁻ CD4 T cells, or CD69⁺CD103⁺ CD4 T cells in lung tissues from IM Prime or Prime and Spike mice 7 and 56 days post boost.

FIG. 6C shows the Measurement of SCV2 spike S1 subunit-specific BALF IgA (H), BALF IgG (I), serum IgA (J), and serum IgG (K) in IM Prime or Prime and Spike mice 7 and 56 days post boost.

FIG. 7A illustrates an experiment in which K18-hACE2 mice were IM primed with 0.05 μg of mRNA-LNP and IN boosted with 1 μg of spike IN 14 days post IM Prime. Six weeks post boost, lung tissues were collected for CD8 T cell analysis by flow cytometry, and BALF and blood were collected for antibody measurement.

FIG. 7B shows the quantification of total Tetramer⁺ CD8 T cells, CD69⁺CD103-Tetramer⁺ CD8 T cells, or CD69⁺CD103⁺Tetramer⁺ CD8 T cells in lung tissues from naïve, IM Prime, or Prime and Spike mice.

FIG. 7C shows measurement of SCV2 spike S1 subunit-specific BALF IgA (E), BALF IgG (F), serum IgA (G), and serum IgG (H) in naïve, IM Prime, or Prime and Spike mice.

FIG. 8A illustrates an experiment in which K18-hACE2 mice were IM primed with 1 μg of mRNA-LNP, followed by boosting with 1 μg of mRNA-LNP IM, or 1 μg of SCV2 spike IN (IN Spike) 14 days post IM Prime. Forty-five days post boost, lung tissues were collected for T cell analysis by flow cytometry, and BALF and blood were collected for antibody measurement.

FIG. 8B shows the quantification of total Tetramer⁺ CD8 T cells, CD69⁺CD103⁻ Tetramer⁺ CD8 T cells, or CD69⁺CD103⁺Tetramer⁺ CD8 T cells in lung tissues from naïve, mRNA-LNP Prime/Boost, or Prime and Spike mice.

FIG. 8C shows Measurement of SARS-CoV-2 spike S1 subunit specific BALF IgA (E), BALF IgG (F), serum IgA (G), and serum IgG (H) in naïve, mRNA-LNP Prime/Boost, or Prime and Spike mice. (I,J) Measurement of neutralization titer against SCV2 spike-pseudotyped VSV.

FIG. 9 shows the length and integrity of extracted mRNA was analyzed using agarose gel electrophoresis. Extracted mRNA was mixed with SYBR Safe stain before being loaded onto a 1% agarose gel, let run in the TAE buffer, and imaged with a gel imaging system.

FIGS. 10A-10B shows (FIG. 10A) Gating strategies to identify extravascular antigen-specific CD8 T cells and polyclonal activated CD4 T cells. (FIG. 10B) Gating strategies to identify extravascular antigen-specific and polyclonal B cell subsets.

### SUMMARY OF THE INVENTION

The invention encompasses a method of enhancing an immune response to an antigen in a human in need thereof, the method comprises administering to the subject an effective amount of a pharmaceutical composition comprising the antigen or a nucleic acid encoding the antigen at a mucosal site, wherein the human has been previously vaccinated against or infected by a virus. In some embodiments, the human has elevated antibodies, memory B cells and effector CD4⁺ and CD8⁺ T cells. In some embodiments, the elevated antibodies, memory B cells and effector CD4⁺ and CD8⁺ T cells are caused by a previous vaccination against a virus.

In some embodiments, the elevated antibodies, memory B cells and effector CD4⁺ and CD8⁺ T cells are caused by a previous infection of a virus.

In some embodiments, the mucosal site is selected from the group consisting of rectal, vaginal, bladder, ocular, oral, sublingual, esophageal, nasal, gastrointestinal, pulmonary and aural mucosal sites.

In some embodiments, the antigen comprises a protein or polypeptide.

In some embodiments, the antigen is multivalent antigen.

In some embodiments, the antigen comprises a nucleic acid encoding a protein or a polypeptide.

In some embodiments, the nucleic acid is DNA or RNA.

In some embodiments, the nucleic acid is mRNA.

In some embodiments, the antigen is derived from a microbial pathogen.

In some embodiments, the microbial pathogen is a mycobacterium, bacterium, fungus, virus, parasite, or prion.

In some embodiments, the virus is selected from the group consisting of rotavirus, norovirus, adenovirus, astrovirus, variants thereof, and any combination thereof.

In some embodiments, the virus is selected from the group consisting of influenza virus, respiratory syncytial virus, parainfluenza viruses, metapneumovirus, rhinovirus, coronavirus, adenovirus, bocavirus, variants thereof, and any combination thereof.

In some embodiments, the virus is selected from the group consisting of herpes simplex virus type 1 (HSV-1), herpes simplex virus type 2 (HSV-2), human papillomavirus (HPV), variants thereof, and any combination thereof.

In some embodiments, the virus is selected from the group consisting of human immunodeficiency virus (HIV), hepatitis A, hepatitis B, hepatitis C, herpes virus, adenovirus, poliomyelitis, Japanese encephalitis, smallpox, influenza virus, flaviviruses, echovirus, rhinovirus, coxsackie virus, coronavirus, respiratory syncytial virus (RSV), mumps virus, rotavirus, measles virus, rubella virus, parvovirus, vaccinia virus, human T-lymphotropic virus (HTLV), dengue virus, human papillomavirus (HPV), molluscum virus, poliovirus, rabies virus, JC virus, arboviral encephalitis virus, SARS-CoV-2, Henoch-Schonlein purpura (HSP), an RNA virus, a DNA virus, variants thereof, and any combination thereof.

In some embodiments, the RNA virus selected from the group consisting of common cold, influenza, SARS, MERS, Covid-19, Dengue Virus, hepatitis C, hepatitis E, West Nile fever, Ebola virus disease, rabies, polio, mumps, measles, variants thereof, and any combination thereof.

In some embodiments, the DNA virus selected from the group consisting of herpes simplex virus, cytomegalo virus, varicella zoster virus, Epstein-Barr virus, roseolo virus, human herpesvirus-7, Kaposi's sarcoma-associated virus, variants thereof, and any combination thereof.

In some embodiments, the pharmaceutical composition is administered by mucosal delivery.

In some embodiments, the mucosal delivery is selected from the group consisting of rectal delivery, buccal delivery, pulmonary delivery, ocular delivery, nasal delivery, intranasal delivery, vaginal delivery and oral delivery.

In some embodiments, the pharmaceutical composition is administered to a mucosal tissue of the human subject.

In some embodiments, the mucosal tissue is selected from the group consisting of anterior nostril, nasal sinus, rectal, vaginal, esophagus, urethral, sublingual and buccal.

In some embodiments, the pharmaceutical composition is administered orally, intravenously, intramuscularly, intradermally, subcutaneously, intranasally, or by inhalation.

In some embodiments, the pharmaceutical composition is administered by intranasal spray.

In some embodiments, the pharmaceutical composition does not comprise an adjuvant.

In some embodiments, the pharmaceutical composition comprises an adjuvant.

In some embodiments, the pharmaceutical composition comprises a lipid nanoparticle (LNP).

In some embodiments, the antigen is encapsulated within the lipid nanoparticle (LNP).

In another aspect, the invention relates to a method of enhancing an immune response to SARS-CoV-2 in a human in need thereof; the method comprises administering to the subject an effective amount of a pharmaceutical composition comprising at least one mRNA at a mucosal site, wherein the human has been previously vaccinated against or infected by SARS-CoV-2. In some embodiments, the mRNA encodes the spike protein of SARS-CoV-2 or a fragment thereof.

In some embodiments, the pharmaceutical composition does not comprise an adjuvant.

In some embodiments, the pharmaceutical composition comprises an adjuvant.

In some embodiments, the pharmaceutical composition further comprises a lipid nanoparticle (LNP).

In some embodiments, the mRNA is encapsulated within the lipid nanoparticle (LNP).

In some embodiments, the lipid nanoparticle (LNP) comprises at least one cationic lipid.

In some embodiments, the at least one cationic lipid comprises 1,2-dimyristoyl-sn-glycero-3-ethylphosphocholine (DMEPC), 1,2-di-O-octadecenyl-3-trimethylammonium propane (DOTMA) and/or 1,2-dioleoyl-3-trimethylammonium propane (DOTAP).

In some embodiments, the lipid nanoparticle (LNP) further comprise at least one phospholipid.

In some embodiments, the at least phospholipid comprises 1,2-distearoyl-sn-glycero-3-phosphocholine (DSPC), 1,2-Dioleoyl-sn-glycero-3-phosphoethanolamine (DOPE), cholesterol (Chol), 1-palmitoyl-2-oleoyl-sn-glycero-3-phosphocholin (POPC) and/or 1,2-dioleoyl-sn-glycero-3-phosphocholine (DOPC).

In some embodiments, the lipid nanoparticle has an average diameter in the range of from about 50 nm to about 1000 nm.

In some embodiments, the lipid nanoparticle has an average diameter in the range of from about 50 nm to about 400 nm, from about 50 nm to about 200 nm, from about 200 nm to about 1000 nm, from about 200 nm to about 800 nm, or from about 300 nm to about 600 nm.

In some embodiments, the immune response is a mucosal immune response.

In some embodiments, the mucosal immune response is an antigen-specific IgA antibody production.

In some embodiments, the mucosal immune response is an antigen-specific IgG antibody production.

In some embodiments, the human has elevated IgA antibody.

In some embodiments, the human has elevated IgG antibody.

### DETAILED DESCRIPTION OF THE INVENTION

### Definition

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The term “variant” means a polypeptide or a nucleotide including an alteration, i.e., a substitution, insertion, and/or deletion, at one or more (e.g., several) positions. In some embodiments, the term “variant” refers to a SARS-CoV-2 virus variant.

The term “spike”, “boost” or “booster” are used interchangeably.

As used herein, the term “immunogenic agent” encompasses any substance, composition of matter, or composition of organic material as for example a suspension of cells or cell components, the immunogenic agent being capable of conferring a substantial immune response to a coronavirus in a human subject, when administered in a suitable amount and in admixture with suitable substances.

The term “immunologically equivalent” means that the polypeptide is functionally equivalent to the polypeptide having the amino acid sequence of any S protein with respect to its ability of eliciting an immune response.

As used herein, the term “polypeptide” encompasses both short peptides with a length of 2-10 amino acid residues, oligopeptides (11-100 amino acid residues), and longer peptides (the usual interpretation of polypeptide, i.e. more than 100 amino acid residues in length) as well as proteins (the functional entity comprising at least one peptide, oligopeptide, or polypeptide which may be chemically modified by glycosylation, or conjugated to other chemical groups). The definition of polypeptides also comprises native forms of polypeptides or proteins in SARS-CoV-2 as well as recombinant proteins or peptides in any type of expression vectors transforming any kind of host, an also chemically synthesized peptides.

The term “nucleic acid” as used herein refers to a polymer containing at least two deoxyribonucleotides or ribonucleotides in either single- or double-stranded form and includes DNA, RNA, and hybrids thereof. DNA may be in the form of antisense molecules, plasmid DNA, cDNA, PCR products, or vectors. RNA may be in the form of small hairpin RNA (shRNA), messenger RNA (mRNA), antisense RNA, miRNA, micRNA, multivalent RNA, dicer substrate RNA or viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid.

The present invention relates to a method of vaccinating a human subject against a virus, wherein the human subject was previously systematically vaccinated against the virus or infected with the virus. The method comprises administering to the subject an effective amount of a pharmaceutical composition comprising an antigen at a mucosal site. This method is also referred as “Prime and Spike” or “Prime and Boost”. In some embodiments, prime and spike method utilizes unadjuvanted intranasal spike boosting that leverages existing immunity generated by primary systematic vaccination to elicit mucosal immune memory within the respiratory tract. Further, using divergent spike proteins, Prime and Spike enables induction of cross-reactive immunity against sarbecoviruses. In some embodiments, Prime and Spike enables multivalent response against sarbecoviruses, such as MERS-CoV, SARS-CoV-1, SARS-Cov-2 or variants thereof.

In one aspect, the invention encompasses a method of enhancing an immune response to an antigen in a human in need thereof, the method comprises administering to the subject an effective amount of a pharmaceutical composition comprising the antigen or a nucleic acid encoding the antigen at a mucosal site, wherein the human has been previously vaccinated against or infected by a virus. In some embodiments, the human has elevated antibodies, memory B cells and effector CD4⁺ and CD8⁺ T cells. In some embodiments, the elevated antibodies, memory B cells and effector CD4⁺ and CD8⁺ T cells are caused by a previous vaccination against a virus. In some embodiments, the previous vaccination is done by parenteral administration.

In some embodiments, the elevated antibodies, memory B cells and effector CD4⁺ and CD8⁺ T cells are caused by a previous infection of a virus. In some embodiments, the elevated antibodies are immunoglobulin G (IgG), IgM, and IgA.

In some embodiments, the mucosal site is selected from the group consisting of rectal, vaginal, bladder, ocular, oral, sublingual, esophageal, nasal, gastrointestinal, pulmonary and aural mucosal sites.

In some embodiments, the antigen comprises a protein or polypeptide.

In some embodiments, the antigen comprises at least one nucleic acid encoding a protein or a polypeptide.

In some embodiments, the nucleic acid is DNA or RNA.

In some embodiments, the nucleic acid is mRNA. In some embodiments, the mRNA is N1-methyl-pseudouridine-modified mRNA. In some embodiments, the mRNA is pseudouridine-modified mRNA. In some embodiments, the antigen comprises two or more different mRNAs. The two or more mRNAs encode two or more different proteins to induce multivalent response.

In some embodiments, the antigen is derived from a microbial pathogen.

In some embodiments, the microbial pathogen is a mycobacterium, bacterium, fungus, virus, parasite, or prion.

In some embodiments, the virus is selected from the group consisting of rotavirus, norovirus, adenovirus, astrovirus, variants thereof, and any combination thereof.

In some embodiments, the virus is selected from the group consisting of influenza virus, respiratory syncytial virus, parainfluenza viruses, metapneumovirus, rhinovirus, coronavirus, adenovirus, bocavirus, variants thereof, and any combination thereof.

In some embodiments, the virus is selected from the group consisting of herpes simplex virus type 1 (HSV-1), herpes simplex virus type 2 (HSV-2), human papillomavirus (HPV), variants thereof, and any combination thereof.

In some embodiments, the virus is selected from the group consisting of human immunodeficiency virus (HIV), hepatitis A, hepatitis B, hepatitis C, herpes virus, adenovirus, poliomyelitis, Japanese encephalitis, smallpox, influenza virus, flaviviruses, echovirus, rhinovirus, coxsackie virus, coronavirus, respiratory syncytial virus (RSV), mumps virus, rotavirus, measles virus, rubella virus, parvovirus, vaccinia virus, human T-lymphotropic virus (HTLV), dengue virus, human papillomavirus (HPV), molluscum virus, poliovirus, rabies virus, JC virus, arboviral encephalitis virus, SARS-CoV-2, Henoch-Schonlein purpura (HSP), an RNA virus, a DNA virus, variants thereof, and any combination thereof.

In some embodiments, the RNA virus selected from the group consisting of common cold, influenza, SARS, MERS, Covid-19, Dengue Virus, hepatitis C, hepatitis E, West Nile fever, Ebola virus disease, rabies, polio, mumps, measles, variants thereof, and any combination thereof.

In some embodiments, the DNA virus selected from the group consisting of herpes simplex virus, cytomegalo virus, varicella zoster virus, Epstein-Barr virus, roseolo virus, human herpesvirus-7, Kaposi's sarcoma-associated virus, variants thereof, and any combination thereof.

In some embodiments, the pharmaceutical composition is administered by mucosal delivery.

In some embodiments, the mucosal delivery is selected from the group consisting of rectal delivery, buccal delivery, pulmonary delivery, ocular delivery, nasal delivery, intranasal delivery, vaginal delivery and oral delivery.

In some embodiments, the pharmaceutical composition is administered to a mucosal tissue of the human subject.

In some embodiments, the mucosal tissue is selected from the group consisting of anterior nostril, nasal sinus, rectal, vaginal, esophagus, urethral, sublingual and buccal.

In some embodiments, the pharmaceutical composition is administered orally, intravenously, intramuscularly, intradermally, subcutaneously, intranasally, or by inhalation.

In some embodiments, the pharmaceutical composition is administered by intranasal spray.

In some embodiments, the pharmaceutical composition does not comprise an adjuvant.

In some embodiments, the pharmaceutical composition comprises an adjuvant.

In some embodiments, the pharmaceutical composition comprises a lipid nanoparticle (LNP). In some embodiments, the lipid nanoparticle (LNP) comprises poly(amine-co-ester) (PACE) polymer. In some embodiments, the PACE polymer are described in U.S. Pat. Nos. 10,682,422; 10,465,042; 9,272,043; 9,895,451; PCT/US2012/067447; and U.S. Patent Publication No. US20200399424, which are incorporated by reference in their entirety.

In some embodiments, the antigen is encapsulated within the lipid nanoparticle (LNP).

In some embodiments, the human has been vaccinated against or infected by the virus about one week ago, two weeks ago, three weeks ago, one month ago, two months ago, three months ago, four months ago, five months ago, six months ago, seven months ago, eight months ago, nine months ago, ten months ago, eleven months ago, or twelve months ago.

In some embodiments, the nucleic acid is RNA. In some embodiments, the RNA is one or more selected from a small RNA, ribozyme, small interfering RNA (siRNA), asymmetrical interfering RNA (aiRNA), microRNA (miRNA), Dicer-substrate RNA (dsRNA), small hairpin RNA (shRNA), transfer RNA (tRNA), messenger RNA (mRNA), and self-amplifying mRNA (SAM). In some embodiments, the nucleic acid is DNA. In some embodiments, the nucleic acid once administered to a human subject would be eventually translated into a protein, wherein the protein effects the therapeutic function or vaccination.

In some embodiments, the nanoparticle comprises one or more compounds described in U.S. Pat. Nos. 10,106,490; 10,723,692; 9,737,619; 9,738,593; and WO2015199952A1, which are incorporated by reference in their entirety.

In some embodiments, the nanoparticle comprises one or more compounds described in U.S. Pat. Nos. 10,682,422; 10,465,042; 9,272,043; 9,895,451; PCT/US2012/067447; and U.S. Patent Publication No. US20200399424, which are incorporated by reference in their entirety.

In some embodiments, the particles have a mean particle size from about 100 nm to about 300 nm, preferably from about 150 nm to about 275 nm. In some embodiments, the weight:weight ratio of polymer:polypeptide is between about 25:1 and 250:1.

Currently approved SARS-CoV-2 mRNA-LNP-based and vector-based vaccines rely on intramuscular administration, which induces high levels of circulating antibodies, memory B cells, and circulating effector CD4⁺ and CD8⁺ T cells in animal models and humans. However, parenteral vaccines do not induce high levels of potent antiviral immune memory at sites of infection such as tissue resident memory T cells (T_{RM}) and B cells (Bm) as well as mucosal IgG and dimeric IgA. This is in contrast to SARS-CoV-2 infection in humans and mice where CD8⁺ T_{RM} are robustly induced. Vaccines targeting the respiratory mucosa could address the shortcomings of parenteral vaccination, as recent preclinical assessments of intranasally delivered SARS-CoV-2 spike encoding adenoviral vectors have shown impressive mucosal immunogenicity as well as protection and reduced viral shedding in mice, hamsters, and nonhuman primates. Preclinical mucosal influenza vaccine studies have also shown that mucosal immunity can enhance protection against heterosubtypic challenge via CD8⁺ T_{RM} or dimeric IgA and may improve durability of immunity.

While primary respiratory administration of vaccines induces potent mucosal immune responses, priming systemically followed by intranasal boosting results in similar systemic immunity, but with the added benefit of enhanced mucosal immunity.

Recent studies have demonstrated decreasing effectiveness of FDA approved mNRA vaccines against VOVID-19 in terms of asymptomatic infection as well as symptomatic and severe infections starting around 4 months post second dose. In the setting of such waning immunity from parenteral vaccination regimens, this disclosure describes a method of enhancing immunity against COVID-19: utilizing the strong systemic priming of mRNA-LNP based vaccine followed by intranasal boosting (IN) boosting with either unadjuvanted spike protein or an immunosilent polyplex encapsulating mRNA.

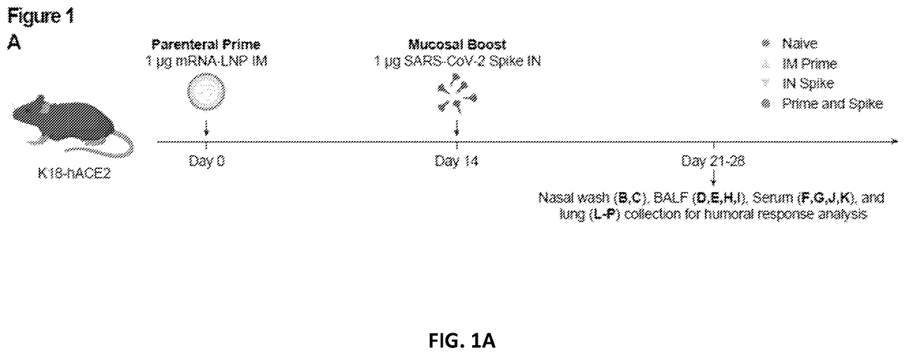

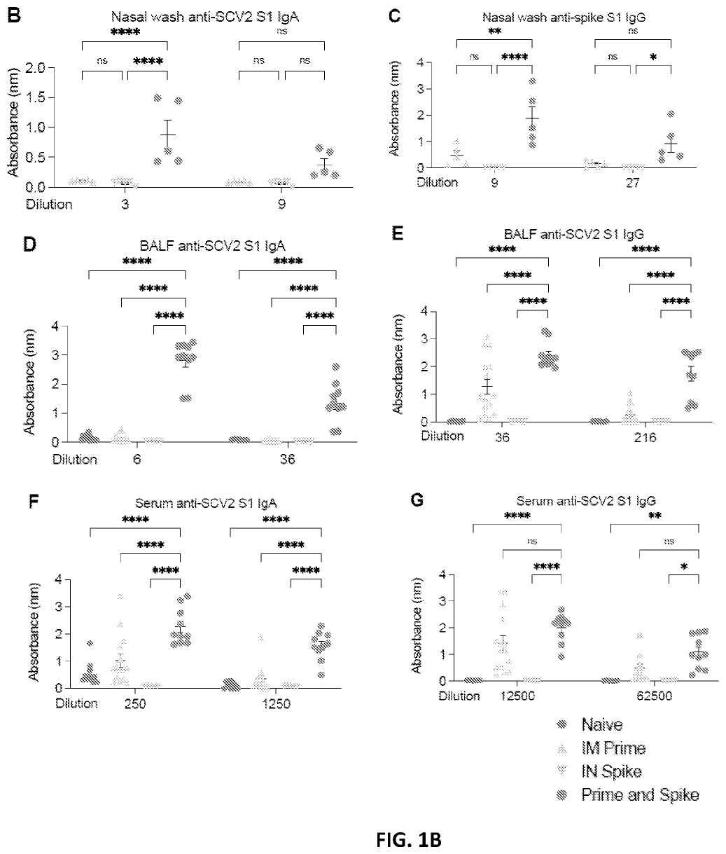

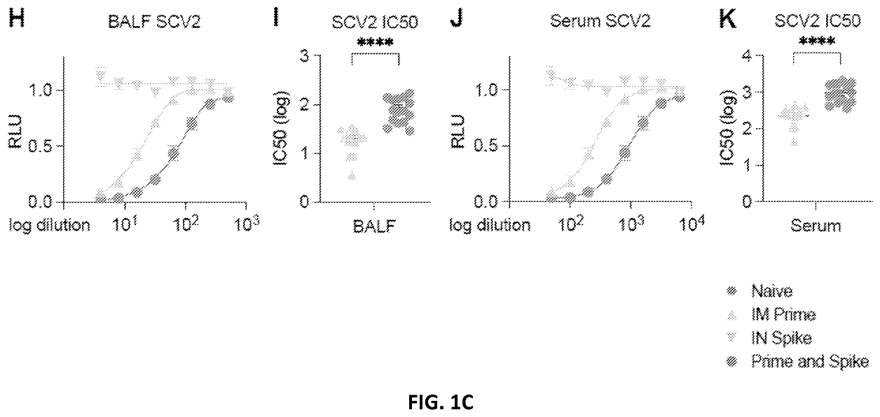

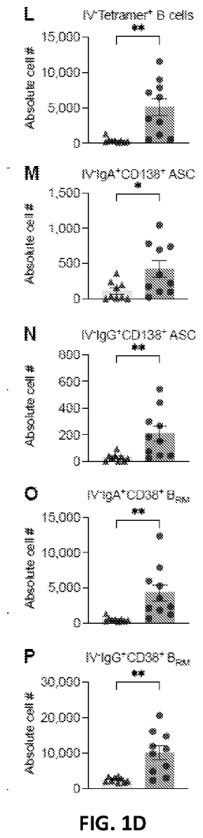

To assess the potential of IN unadjuvanted subunit vaccine boosting for the development of respiratory tract mucosal immunity, K18-hACE2 mice were vaccinated with 1 μg of mRNA-LNP (Comirnaty) by IM injection (Prime), followed 14 days later by 1 μg of recombinant unadjuvanted spike protein by IN administration (Prime and Spike). Additional control groups include K18-hACE2 mice that received IM Prime only and mice that received IN spike only at boosting. Mice were euthanized at day 21 or 28 (7- or 14-days post boosting) and assessed for the development of mucosal humoral immunity (FIG. 1A). Anti-SARS-CoV-2 spike S1 IgG and IgA in nasal wash, bronchoalveolar lavage fluid (BALF), and serum were assessed. Only mice that received Prime and Spike developed high levels of anti-SARS-CoV-2 IgA and IgG in the nasal wash and BALF (FIG. 1B(B-E)). Neither IM Prime only nor IN spike only was sufficient for the development of mucosal antibodies. In the serum, IM Prime only was sufficient to induce low levels of IgA and IgG; however, Prime and Spike led to significant systemic boosting of both anti-spike S1 IgA and IgG (FIG. 1B (F,G)). These increases in antibody level correlated with increases in neutralization titers both in the BALF and serum (FIG. 1B (H-K)). These results indicate that single-dose unadjuvanted intranasal spike alone is not immunogenic, and that induction of a potent mucosal and systemic antibody response by unadjuvanted spike requires prior systemic priming, in this case by mRNA-LNP.

Using intravenous (IV) CD45 labeling combined with B cell tetramers specific for receptor binding domain (RBD) of the spike protein, it was found that Prime and Spike leads to increased antigen specific B cells within lung tissue (IV-CD19⁺B220⁺Tetramer⁺) (FIG. 1D (L)). Given that the tetramer only assessed for RBD binding, it was also looked at the polyclonal tissue response which likely represents a more complete set of B cells reactive to the entire spike within lung tissue. It was found increases in class switched antibody secreting cells (ASC) (IV-CD19^{+/-}CD138⁺) in lung tissue expressing IgA or IgG (FIG. 1D(M,N)), and it was found increased class switched Bm (IV⁻CD19⁺B220⁺IgD⁻IgM⁻CD38⁺) expressing IgA or IgG (FIG. 1D (O,P)). These results are consistent with increased mucosal antibody production and indicate that Prime and Spike elicits local B cell responses in the lung.

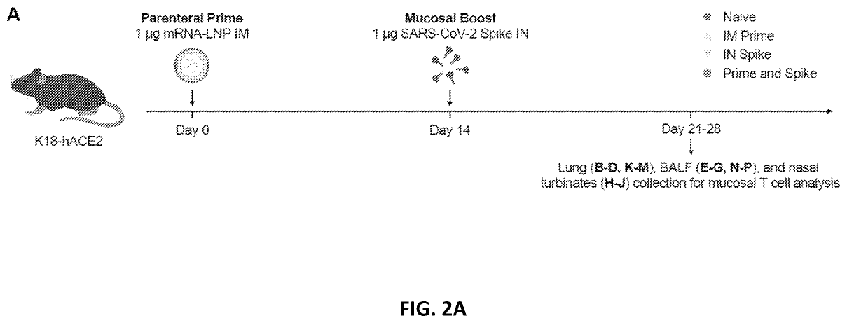

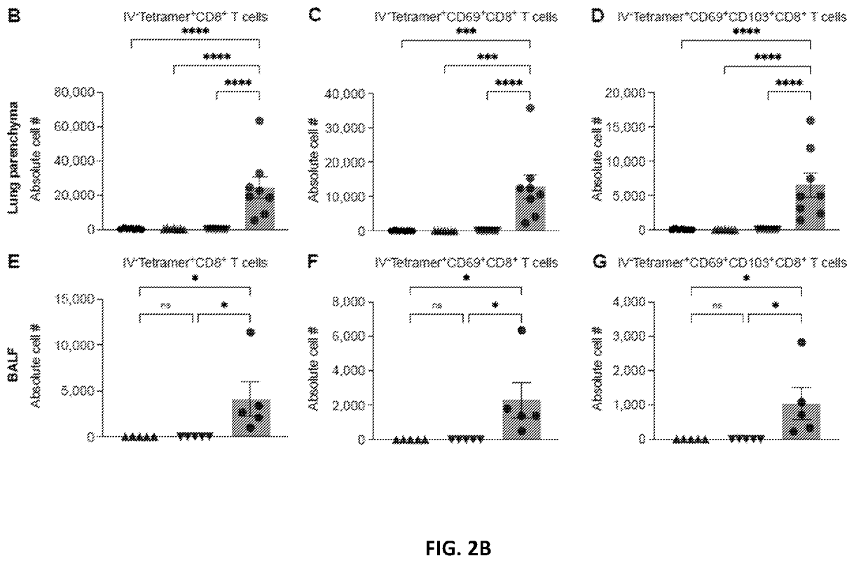

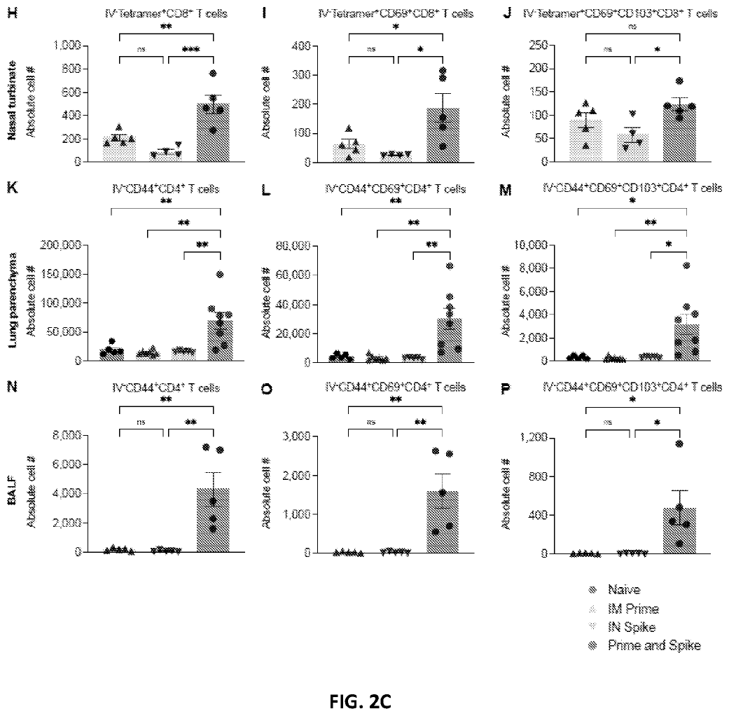

Similar to above, CD45 IV labeling to differentiate circulating from immune cells within lung tissue was combined with major histocompatibility complex (MHC) class I tetramer to a conserved sarbecovirus spike epitope (VNFNFNGL). It was found significant induction of spike IV⁻ tetramer⁺ CD8⁺ T cells, which expressed canonical markers of T_{RM} including CD69⁺ and CD103⁺, within lung tissue (FIG. 2B (B-D)), the lower airway BALF (FIG. 2B (E-G)), and in the upper airway nasal turbinate (FIG. 2C (H-J)). Additionally, it was found significant increases in antigen experienced CD4⁺ T cells (IVCD44⁺CD4⁺), many of which also expressed markers of T_{RM} CD69⁺ and CD103⁺ both within lung tissue (FIG. 2C (K-M)) and from lower airway recovered from BALF (FIG. 2C (N-P)). These results indicate that Prime and Spike not only induces humoral mucosal responses, but also robustly elicits lung parenchyma and airway CD8⁺ T_{RM} and CD4⁺ T_{RM}.

K18-hACE2 mice that received 1 μg IM Prime were boosted with IN Spike 84 days later. We sampled humoral and cellular mucosal immune responses at day 91 (7 days post boost) and day 140 (56 days post boost). We found that delayed IN Spike was sufficient to induce CD8⁺ T_{RM} which persisted for at least 56 days. CD4⁺ T_{RM} were induced early at 7 days post boost; however, their longevity seemed to wane by 56 days, at least polyclonally. Similar to the CD8⁺ T_{RM} response, it was found not only adequate humoral response to delayed boosting, but strong and increasing mucosal IgA and IgG in BALF, and strong and increasing serum IgA and IgG at 56 days post boosting. These results indicate that Prime and Spike given with a 3-month interval between doses is sufficient to elicit long lasting mucosal and systemic humoral and cellular immune responses.

In another aspect, the invention relates to a method of enhancing an immune response to SARS-CoV-2 in a human in need thereof; the method comprises administering to the subject an effective amount of a pharmaceutical composition comprising at least one mRNA at a mucosal site, wherein the human has been previously vaccinated against or infected by SARS-CoV-2. In some embodiments, the mRNA encodes the spike protein of SARS-CoV-2 or a fragment thereof.

In some embodiments, the human has been previously vaccinated with one or more COVID-19 vaccines selected from the group consisting of BNT162b2 (Pfizer/BioNTech), mRNA-1273 (Moderna), AZD1222/ChAdOxl (AstraZeneca/Oxford Univ), Ad5-vectored COVID-19 vaccine (CanSino Biologies), CoronaVac (Sinovac), NVX-CoV2373 (Novavax), and combinations thereof.

In some embodiments, the human has elevated IgG antibody caused by a previous vaccination against MERS-CoV, SARS-CoV-1, SARS-Cov-2 or variants thereof.

In some embodiments, the human has elevated IgM antibody caused by a previous vaccination against MERS-CoV, SARS-CoV-1, SARS-Cov-2 or variants thereof.

In some embodiments, the human the human has elevated IgA antibody caused by a previous vaccination against MERS-CoV, SARS-CoV-1, SARS-Cov-2 or variants thereof.

In some embodiments, the human has elevated IgG antibody caused by a previous infection of MERS-CoV, SARS-CoV-1, SARS-Cov-2 or variants thereof.

In some embodiments, the human has elevated IgM antibody caused by a previous infection of MERS-CoV, SARS-CoV-1, SARS-Cov-2 or variants thereof.

In some embodiments, the human has elevated IgA antibody caused by a previous infection of MERS-CoV, SARS-CoV-1, SARS-Cov-2 or variants thereof.

In some embodiments, the elevated IgG is in a range of about 100-150, about 100-200, about 100-300, about 100-400, about 150-200, about 150-250, about 150-300, about 150-400, about 200-250, about 200-300, about 200-350, or about 200-400 BAU/ml.

In some embodiments, the elevated IgG is about 150, 155, 160, 165, 170, 175, 180, 185, 190, 195, 200, 205, 210, 215, 220, 225, 230, 235, 240, 245, 250, 255, 260, 265, 270, 280, 290, 295, or 300 BAU/ml.

In some embodiments, the elevated IgM is in a range of about 25-100, about 25-150, about 25-200, about 25-300, about 50-100, about 50-150, about 50-200, about 50-300, about 75-100, about 75-150, about 75-200, about 75-300, about 100-150, about 100-200, about 100-300, about 125-200, about 125-300, about 150-200, about 150-300, about 200-300, about 250-300 AU/ml.

In some embodiments, the elevated IgM is about 50, 55, 60, 65, 70, 75, 80, 85, 90, 95, 100, 105, 110, 115, 120, 125, 130, 135, 140, 145, 150, 155, 160, 165, 170, 180, 190, 195, or 200 AU/ml.

In some embodiments, the elevated IgA is in a range of about 10-100, about 10-150, about 10-200, about 25-100, about 25-150, about 25-200, about 50-100, about 50-150, about 50-200, about 75-100, about 75-150, about 75-200, about 100-150, about 100-200, about 125-150, about 125-200, about 150-200 AU/ml.

In some embodiments, the elevated IgA is about 25, 30, 35, 40, 45, 50, 55, 60, 65, 70, 75, 80, 85, 90, 95, 100, 105, 110, 115, 120, 125, 130, 135, 140, 145, 150 AU/ml.

In some embodiments, the at least one mRNA encodes the spike protein of SARS-CoV-2 or variants thereof or a fragment thereof. In some embodiments, the at least one mRNA is a multivalent antigen. In some embodiments, the pharmaceutical composition comprises two or more different mRNAs. The two or more mRNAs encode two or more different proteins to induce multivalent response against SARS-CoV-2. In some embodiments, the mRNA is N1-methyl-pseudouridine-modified mRNA. In some embodiments, the mRNA is pseudouridine-modified mRNA.

In some embodiments, the pharmaceutical composition does not comprise an adjuvant.

In some embodiments, the pharmaceutical composition comprises an adjuvant.

In some embodiments, the pharmaceutical composition further comprises a lipid nanoparticle (LNP). In some embodiments, the lipid nanoparticle (LNP) comprises poly(amine-co-ester) (PACE) polymer.

In some embodiments, the at least one mRNA is encapsulated within the lipid nanoparticle (LNP).

In some embodiments, the lipid nanoparticle (LNP) comprises at least one cationic lipid.

In some embodiments, the at least one cationic lipid comprises 1,2-dimyristoyl-sn-glycero-3-ethylphosphocholine (DMEPC), 1,2-di-O-octadecenyl-3-trimethylammonium propane (DOTMA) and/or 1,2-dioleoyl-3-trimethylammonium propane (DOTAP).

In some embodiments, the lipid nanoparticle (LNP) further comprise at least one phospholipid.

In some embodiments, the at least phospholipid comprises 1,2-distearoyl-sn-glycero-3-phosphocholine (DSPC), 1,2-Dioleoyl-sn-glycero-3-phosphoethanolamine (DOPE), cholesterol (Chol), 1-palmitoyl-2-oleoyl-sn-glycero-3-phosphocholin (POPC) and/or 1,2-dioleoyl-sn-glycero-3-phosphocholine (DOPC).

In some embodiments, the lipid nanoparticle has an average diameter in the range of from about 50 nm to about 1000 nm.

In some embodiments, the lipid nanoparticle has an average diameter in the range of from about 50 nm to about 400 nm, from about 50 nm to about 200 nm, from about 200 nm to about 1000 nm, from about 200 nm to about 800 nm, or from about 300 nm to about 600 nm.

In some embodiments, the immune response is a mucosal immune response.

In some embodiments, the mucosal immune response is an antigen-specific IgA antibody production.

In some embodiments, the human has been vaccinated against or infected by the virus about one week ago, two weeks ago, three weeks ago, one month ago, two months ago, three months ago, four months ago, five months ago, six months ago, seven months ago, eight months ago, nine months ago, ten months ago, eleven months ago, or twelve months ago.

In some embodiments, the pharmaceutical composition described herein comprises a polypeptide as an antigen for vaccinating a human subject against SARS-CoV-2 and an immunogenic variant thereof. In some embodiments, the polypeptide is a coronavirus spike (S) protein, an immunogenic variant thereof, or an antigenic fragment thereof. In some embodiments, the polypeptide has an amino acid sequence which has a degree of sequence identity of at least 85%, 86%, 87%, 88%, 89%, 90%, 91%, 92%, 93%, 94%, 95%, 96%, 97%, 98%, or 99%, with any known S protein, or a subunit or fragment thereof. In some embodiments, the polypeptide has an amino acid sequence which has a degree of sequence identity of 85%, 86%, 87%, 88%, 89%, 90%, 91%, 92%, 93%, 94%, 95%, 96%, 97%, 98%, or 99%, with any known S protein, or a subunit or fragment thereof. In some embodiments, the variants are SARS-CoV-2 Spike protein variants found in different strains of SARS-CoV-2. Variants include, but are not limited to, Spike proteins from the alpha, beta or delta variant of SARS-CoV-2, B.1.1.7 strain, B.1.351 strain, P.1 strain, CAL 20 strain or any combination thereof.

In some embodiments, SARS-CoV-2 variants include, but are not limited to, Alpha (B.1.1.7 and Q lineages), Beta (B.1.351 and descendent lineages), Gamma (P.1 and descendent lineages), Delta (B.1.617.2 and AY lineages), Epsilon (B.1.427 and B.1.429), Eta (B.1.525), Iota (B.1.526), Kappa (B.1.617.1), 1.617.3, Mu (B.1.621, B.1.621.1), Zeta (P.2), Mu (B.1.621, B.1.621.1), Omicron (Pango lineages B.1.1.529, BA.1, BA.1.1, BA.2, BA.3), and combinations thereof.

The DNA sequence encoding the Spike protein of SARS-CoV-2 isolated in Wuhan is listed as SEQ ID NO: 1.

Exemplary variants of the spike protein from different strains are set forth in Table 1.

Table 1. List of amino acid positions and relative amino acid changes in the different variants in the Spike protein with respect to the ancestral Wuhan strain Spike protein (SEQ ID NO: 2).

| protein | position | Wuhan | B1.1.7 | B1.351 | P.1 | CAL.20 |
|---|---|---|---|---|---|---|
| S | 13 | S | | | | I |
| S | 18 | L | | F | F | |
| S | 20 | T | | | N | |
| S | 26 | P | | | S | |
| S | 69 | H | Del | | | |
| S | 70 | V | Del | | | |
| S | 80 | D | | A | | |
| S | 138 | D | | | Y | |
| S | 145 | Y | Del | | | |
| S | 152 | W | | | | C |
| S | 190 | R | | | S | |
| S | 215 | D | | G/H | | |
| S | 241 | L | | Del | | |
| S | 242 | L | | Del | | |
| S | 243 | A | | Del | | |
| S | 417 | K | | N | T | |
| S | 452 | L | | | | R |
| S | 484 | E | | K | K | |
| S | 501 | N | Y | Y | Y | |
| S | 570 | A | D | | | |
| S | 614 | D | G | G | G | G |
| S | 655 | H | | | Y | |
| S | 681 | P | H | | | |
| S | 701 | A | | V | | |
| S | 716 | T | I | | | |
| S | 938 | L | | | | F |
| S | 982 | S | A | | | |
| S | 1027 | T | | | I | |
| S | 1118 | D | H | | | |
| S | 1176 | V | | | F | |
| S | 1191 | K | | | | N |

In some embodiments, the polypeptide comprises other amino acid sequences for strains of SARS-CoV-2 S protein include any of those disclose in Deng (2020) Science, 8:eabb9263 and Taboada (2020) J. Virol. 94:e01056. However, it is highly likely that other SARS-CoV-2 strains will exhibit substantially the same immunological properties as the alpha variant S protein, fragments and subunits thereof from such strains. In some embodiments, the polypeptide is selected from the group consisting of the M protein, E protein, N protein from SARS-CoV-2 or a variant thereof, and combinations thereof.

In some embodiments, the S protein variant described herein comprises a mutation at a position corresponding to position 501N in the alpha variant. In some embodiments, the amino acid corresponding to position 501N is substituted with Y. In some embodiments, the S protein variant described herein comprising a mutation at a position corresponding to position 501N may comprise one or more further mutations. Such one or more further mutations may be one or more selected from mutations at positions corresponding to the following amino acids at positions relative to the alpha variant: 18L, 69H, 70V, 80D, 144Y, 215D, 246R, 242L, 243A, and 244L, 417K, 484E, 570A, 614D, 681P, 701A, 716T, 982S, and 1118D. In some embodiments, the amino acid corresponding to position 69H in the alpha variant is deleted. In some embodiments, the amino acid corresponding to position 70V in is deleted. In some embodiments, the amino acid corresponding to position 144Y is deleted. In some embodiments, the amino acid corresponding to position 570A in is D. In some embodiments, the amino acid corresponding to position 614D is G. In some embodiments, the amino acid corresponding to position 681P is H. In some embodiments, the amino acid corresponding to position 716T is I. In some embodiments, the amino acid corresponding to position 982S is A. In some embodiments, the amino acid corresponding to position 1118D is H. In some embodiments, the amino acid corresponding to position 80D is A. In some embodiments, the amino acid corresponding to position 215D is G. In some embodiments, the amino acid corresponding to position 484E is K. In some embodiments, the amino acid corresponding to position 701A is V. In some embodiments, the amino acid corresponding to position 18L is F. In some embodiments, the amino acid corresponding to position 246R is I. In some embodiments, the amino acid corresponding to position 417K is N. In some embodiments, the amino acid corresponding to position 242L is deleted. In some embodiments, the amino acid corresponding to position 243A is deleted. In some embodiments, the amino acid corresponding to position 244L is deleted.

In some embodiments, the S protein variant described herein is the S protein of SARS-CoV-2 delta. In some embodiments, the S protein of SARS-CoV-2 delta has the following spike protein substitutions relative to the alpha variant: T19R, V70F, T95I, G142D, 156E deletion, 157F deletion, R158G, A222V, W258L, K417N, L452R, T478K, D614G, P681R, and D950N.

In some embodiments, the S protein variant described herein is the S protein of SARS-CoV-2 omicron. In some embodiments, the S protein of SARS-CoV-2 omicron has the following spike protein substitutions relative to the alpha variant: A67V, deletion of amino acids 69-70, T95I, deletion of amino acids 142-144, Y145D, deletion of amino acid 211, L212I, insertion of amino acids EPE at 214, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F.

In some embodiments, administration of the pharmaceutical composition described herein may be performed by single administration or boosted by multiple administrations.

In some embodiment, pharmaceutical compositions described herein may be administered intravenously, intraarterially, subcutaneously, intradermally or intramuscularly. In some embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection.

In some embodiments, pharmaceutical compositions described herein may be administered intranasally.

In some embodiments, an amount the polypeptide described herein from 0.1 μg to 300 μg, 0.5 μg to 200 μg, or 1 μg to 100 μg, such as about 1 μg, about 3 μg, about 10 μg, about μg, about 50 μg, or about 100 μg may be administered per dose. In some embodiments, the invention envisions administration of a single dose. In some embodiments, the invention envisions administration of a priming dose followed by one or more booster doses. The booster dose or the first booster dose may be administered about one week, about two weeks, about three weeks, about four weeks, or about five weeks following administration of the priming dose. In some embodiments, the booster dose or the first booster dose may be administered about one month, about two months, about three months, about four months, or about five months, about six months, about seven months, about eight months, about nine months, about ten months, about eleven months, or about twelve months following administration of the priming dose.

In some embodiments, an amount of the polypeptide described herein of 60 μg or lower, 50 μg or lower, 40 μg or lower, 30 μg or lower, 20 μg or lower, 10 μg or lower, 5 μg or lower, 2.5 μg or lower, or 1 μg or lower may be administered per dose.

In some embodiments, an amount of the polypeptide described herein of at least 0.25 μg, at least 0.5 μg, at least 1 μg, at least 2 μg, at least 3 μg, at least 4 μg, at least 5 μg, at least 10 μg, at least 20 μg, at least 30 μg, or at least 40 μg may be administered per dose.

In some embodiments, an amount of the polypeptide described herein of 0.25 μg to 60 μg, 0.5 μg to 55 μg, 1 μg to 50 μg, 5 μg to 40 μg, or 10 μg to 30 μg may be administered per dose.

The pharmaceutical compositions and products described herein may be provided as a frozen concentrate for solution for injection, e.g., at a concentration of 0.50 mg/mL. In some embodiments, for preparation of solution for injection, a drug product is thawed and diluted with isotonic sodium chloride solution (e.g., 0.9% NaCl, saline), e.g., by a one-step dilution process. In some embodiments, bacteriostatic sodium chloride solution (e.g., 0.9% NaCl, saline) cannot be used as a diluent. In some embodiments, a diluted drug product is an off-white suspension. The concentration of the final solution for injection varies depending on the respective dose level to be administered.

The invention also encompasses a kit comprising the pharmaceutical composition described herein and means for administration. In some embodiments, the kit comprises a nasal spray device for intranasal administration. Nasal spray devices are well known in the art and described in Djupesland, Drug Deliv. Transl. Res. (2013) 3(1): 42-62 which is incorporated by reference in its entirety. The kit may be convenient for self administration for vaccinating against SARS-CoV-2 or a variant thereof.

In the kit according to the invention the pharmaceutical composition comprises 0.5 to 75 μg of the polypeptide, such as 0.5 to 50 μg of the polypeptide, or 5 to 50 μg of the polypeptide.

### EXAMPLES

The present teachings, having been generally described, will be more readily understood by reference to the following examples, which are included for the purposes of illustrating certain aspects and embodiments of the present disclosure.

### Methods and Materials:

All procedures were performed in a BSL-3 facility (for SARS-CoV-2-infected mice) with approval from the Yale Institutional Animal Care and Use Committee and Yale Environmental Health and Safety.

### Cell and Virus

Vero E6 cells over expressing hACE2 and TMPRSS2 (kindly provided by Barney Graham NIH-VRC) were cultured in Dulbecco's Modified Eagle Medium (DMEM) supplemented with 1% sodium pyruvate and 5% fetal bovine serum (FBS) at 37° C. and 5% CO2. SARS-CoV-2 isolate hCOV-19/USA-WA1/2020 (NR-52281) was obtained from BEI Resources and was amplified in VeroE6 cells over expressing hACE2 and TMPRSS2. Cells were infected at a MOI 0.01 for two-three days to generate a working stock and after incubation the supernatant was clarified by centrifugation (500 μg×5 min) and filtered through a 0.45-micron filter and stored at −80° C. Viral titers were measured by standard plaque assay using Vero E6 cells over expressing hACE2 and TMPRSS2.

### Mice

B6.Cg-Tg(K18-ACE2)2Prlmn/J (K18-hACE2) mice were purchased from The Jackson Laboratory and subsequently bred and housed at Yale University. Eight to twelve-week-old female were used for immunization experiments. All procedures used in this study (sexmatched, age-matched) complied with federal guidelines and the institutional policies of the Yale School of Medicine Animal Care and Use Committee.

### Example 1: SARS-CoV-2 Infection

Mice were anesthetized using 30% v/v Isoflurane diluted in propylene glycol. Using a pipette, 50 μL containing 6×10⁵ PFU SARS-CoV-2 was delivered intranasally.

### Example 2: mRNA Extraction from Comirnaty mRNA-LNP

mRNA was extracted from the vaccine formulation with a TRIzol/chloroform separation method described here. Briefly, aliquots of vaccine were dissolved in TRIzol LS (Thermo Fisher Scientific) at 1:6.6 vaccine to TRIzol volume ratio. Following a 15 min incubation (37° C., shaking) 0.2 mL of chloroform was added per 1 mL of TRIzol. The solution was shaken vigorously for 1 min and then incubated at room temperature for 3 min. The solution was centrifuged at 12,000×g for 8 min at 4° C. The aqueous layer containing the isolated mRNA was further purified with a RNeasy Maxi Kit purchased from Qiagen (Germantown, MD, USA) following the manufacturers protocol. The RNA was eluted from the column on the final step with sodium acetate buffer (25 mM, pH 5.8) warmed to 37° C. Extracted mRNA was analyzed for concentration and purity by NanoDrop measurements of the absorbance at 260, 280 and 230 nm, with purity being assessed as A260/A280>2 and A260/A230>2. Agarose gel electrophoresis was used to determine the length and verify that the mRNA remained intact. Extracted mRNA containing 1:100 SYBR Safe stain (Thermo Fisher Scientific) was loaded onto a 1% agarose gel and run at 75V with TAE buffer containing 1:5000 SYBR Safe stain.

### Example 3: PACE Polyplex Formulation and Characterization

PACE polymers were synthesized and characterized as previously described. All polyplexes were formulated at a 50:1 weight ratio of polymer to mRNA. PACE polymers were dissolved at 100 mg/mL overnight in DMSO (37° C., shaking). Prior to polyplex fabrication, an optimal PACE polymer blend was produced by mixing solutions of PACE polymers containing an end-group modification and a polyethylene glycol tail. mRNA and polymer were diluted into equal volumes of sodium acetate buffer (25 mM, pH 5.8). The polymer dilution was then vortexed for 15 s, mixed with the mRNA dilution, and vortexed for an additional 25 s. Polyplexes were incubated at room temperature for 10 min before use.

### Example 4: Vaccination

Used vials of Comirnaty vaccine were acquired from Yale Health pharmacy within 24 hr of opening and stored at 4° C. Vials contained residual vaccine (diluted to 100 μg/mL per manufacturer's instructions) which was removed with spinal syringe and pooled. Pooled residual vaccine was aliquoted and stored at −80° C. Mice were anaesthetized using a mixture of ketamine (50 mg/kg) and xylazine (5 mg/kg), injected intraperitoneally. Vaccine was diluted in sterile PBS and 10 μL or 20 μL was injected into the left quadriceps muscle with a 31 μg syringe for a final dose of 1 μg or 0.05 μg as indicated. For intranasal vaccination with SARS-CoV-2 stabilized spike (ACRO biosystems, SPN-C52H9) or SARS-CoV-1 spike (ACRO biosystems, SPN-S52H6) was reconstituted in sterile endotoxin free water according to the manufacturers protocol, and then diluted in sterile PBS and stored at −80° C. Mice were anesthetized using 30% v/v Isoflurane diluted in propylene glycol and administered 1 μg or 5 μg (as indicated) in 50 μL via the IN route. For IN mRNA-PACE, 50 μL of polyplexes in solution was given at the indicated dose.

### Example 5: Viral Titer Analysis

At indicated time points mice were euthanized in 100% Isoflurane. ˜50% of total lung was placed in a bead homogenizer tube with 1 mL of PBS with 2% FBS and 2% antibiotics/antimycotics (Gibco) and stored at −80° C. Lung homogenates were cleared of debris by centrifugation (3900 rpm for 10 min). Infectious titers of SARS-CoV-2 were determined by plaque assay in VeroE6 cells over expressing hACE2 and TMPRSS2 in DMEM supplemented with NaHCO3, 2% FBS, and 0.6% Avicel RC-581. Plaques were resolved at 40-42 hours post infection by fixing in 10% Neutral Buffered Formalin for 1 hour followed by staining for 1 hour in 0.5% crystal violet in 20% ethanol for 30 min. Plates were rinsed in water to visualize plaques.

### Example 6: SARS-CoV-2 Specific-Antibody Measurements

ELISAs were performed as previously described with modifications noted and reproduced here for convenience. 96-well MaxiSorp plates (Thermo Scientific #442404) were coated with 50 L/well of recombinant SARS-CoV-2 S1 protein (ACRO Biosystems S1NC52H3) or SARS-CoV-1 S1 protein (ACRO Biosystems S1N-S52H5) at a concentration of 2 μg/mL in PBS and were incubated overnight at 4° C. The coating buffer was removed, and plates were incubated for 1 hour at RT with 250 μL of blocking solution (PBS with 0.1% Tween-20, 5% milk powder). Serum or bronchoalveolar lavage fluid (BALF) was diluted in dilution solution (PBS with 0.1% Tween-20 and 2% milk powder) and 100 L of diluted serum or BALF was added and incubated for two hours at RT. Plates were washed five times with PBS-T (PBS with 0.05% Tween-20) with automatic plate washer (250 μL per cycle) and 50 μL of HRP antimouse IgG (Cell Signaling Technology #7076, 1:3,000) or HRP anti-mouse IgA (Southern Biotech #1040-05, 1:1,000) diluted in dilution solution added to each well. After 1 h of incubation at RT, plates were washed three times with PBS-T in automatic plate washer. Plates were developed with 50 μL of TMB Substrate Reagent Set (BD Biosciences #555214) and the reaction was stopped after 15 min by the addition of 50 μL 2 N sulfuric acid. Plates were then read at a wavelength of 450 nm and 570 nm, and the difference reported.

### Example 7: Immunohistochemistry and Pathological Analysis

Yale pathology performed embedding, sectioning, and H&E staining of lung tissue. A pulmonary pathologist reviewed the slides blinded and identified immune cell infiltration and other related pathologies. Scoring 1-4 as follows: (1) Mild patchy mononuclear infiltrate, parenchymal and perivascular, with variably reactive pneumocytes and stromal rection; (2) Moderate patchy mononuclear infiltrate, parenchymal and perivascular, with variably reactive pneumocytes and stromal rection; (3) Mild, dense mixed infiltrate including mononuclear cells and granulocytes/neutrophils; (4) Moderate, dense mixed infiltrate including mononuclear cells and granulocytes/neutrophils.

### Example 8: Intravascular Labeling, Cell Isolation, and Flow Cytometry

To discriminate intravascular from extravascular cells, mice were anesthetized with 30% Isoflurane and injected i.v. with APC/Fire 750 CD45 Ab (30-F11, AB_2572116, BioLegend, #103154) and after 3 min labeling, mice were euthanized. Lungs were minced with scissors and incubated in a digestion cocktail containing 1 mg/mL collagenase A (Roche) and 30 g/mL DNase I (Sigma-Aldrich) in RPMI at 37° C. for 45 min. Tissue was then filtered through a 70-μm filter. Cells were treated with ammonium-chloride-potassium buffer and resuspended in PBS with 1% BSA. Single cell suspensions were incubated at 4° C. with Fc block and Aqua cell viability dye for 20 min. Cells were washed once with PBS before surface staining. For T cell analysis, cells were stained with anti-CD103 (BV421, 2E7, AB_2562901, BioLegend #121422), anti-CD3 (BV605, 17A2, AB_2562039, BioLegend #100237), anti-CD44 (BV711, IM7, AB 2564214, BioLegend #103057), anti-CD62L (FITC, MEL-14, AB_313093, BioLegend #104406), anti-CD8a (PerCP/Cy5.5, 16-10A1, AB_2566491, BioLegend #305232), anti-CD69 (PE/Cy7, H1.2F3, AB_493564, BioLegend #104512), anti-CD183 (CXCR3) (APC, CXCR3-173, AB_1088993, BioLegend #126512), anti-CD4 (AF700, GK 1.5, AB_493699, BioLegend #100430), and PESARS-CoV-2 S 539-546 MHC class I tetramer (H-2K(b)) for 30 min at 4° C. For B cell analysis, cells were stained with ant-GL7 (Pacific Blue, GL7, AB_2563292, BioLegend #144614), anti-IgM (BV605, RMM-1, AB_2563358, BioLegend #406523), anti-CD138 (BV711, 281-2, AB_2562571, BioLegend #142519), anti-CD19 (BV785, 6D5, AB_11218994, BioLegend #115543), anti-IgA (FITC, polyclonal, AB_2794370, SouthernBiotech #1040-02), anti-B220 (PerCP/Cy5.5, RA3-6B2, AB_893354, BioLegend #103236), PE-SARS-CoV-2 RBD tetramer, anti-CD38 (PE/Cy7, 90, AB_2275531, BioLegend #102718), APC-SARS-CoV-2 RBD tetramer, and anti-IgD (AF700, 11-26c.2a, AB_2563341, BioLegend #405730) for 30 min at 4° C. After washing with PBS, cells were fixed using 4% paraformaldehyde. Cell population data were acquired on an Attune NxT Flow Cytometer and analyzed using FlowJo Software (10.5.3; Tree Star).

### Example 9: SARS-CoV-2 Receptor Binding Domain B Cell Tetramer Production

Recombinant SARS-CoV-2 Spike RBD His Biotin Protein, CF (R&D/BT10500-050) was incubated at a 4:1 molar ratio with either streptavidin-PE (Prozyme PJRS25) or streptavidin-APC (Prozyme PJ27S) for 30 min at 4° C. Mixture was then purified and concentrated in an Amicon Ultra (50 kDA MWCO) spin column and washed 1× with sterile cold PBS. Concentration was determined on a nanodrop and using fluorophore specific absorbance and tetramers were diluted to 1.0 μM in PBS and stored at 4° C.

### Example 10: Pseudovirus Production

VSV-based pseudotyped viruses were produced as previously described. Vector pCAGGS containing the SARS-CoV-2 Wuhan-Hu-1 spike glycoprotein gene was produced under HHSN272201400008C and obtained through BEI Resources (NR-52310). The sequence of the Wuhan-Hu-1 isolate spike glycoprotein is identical to that of the USA-WA1/2020 isolate. SARS-CoV-1 Spike encoding plasmid was kindly provided by Dr. Vincent Munster and previously described. 293T cells were transfected with either spike plasmid, followed by inoculation with replication-deficient VSV-expressing Renilla luciferase for 1 hour at 37° C. The virus inoculum was then removed, and cells were washed three times with warmed PBS. The supernatant containing pseudovirus was collected 24 and 48 hours after inoculation, clarified by centrifugation, concentrated with Amicon Ultra centrifugal filter units (100 kDa), and stored in aliquots at −80° C. Pseudoviruses were titrated in Huh7.5 cells to achieve a relative light unit signal of ˜600 times the cell-only control background.

### Example 11: Pseudovirus Neutralization Assay

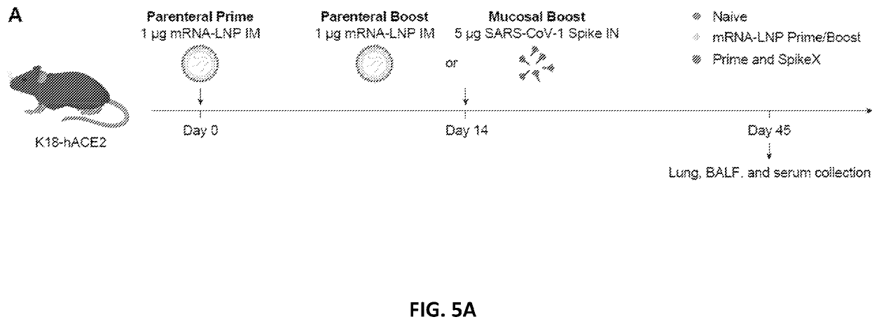

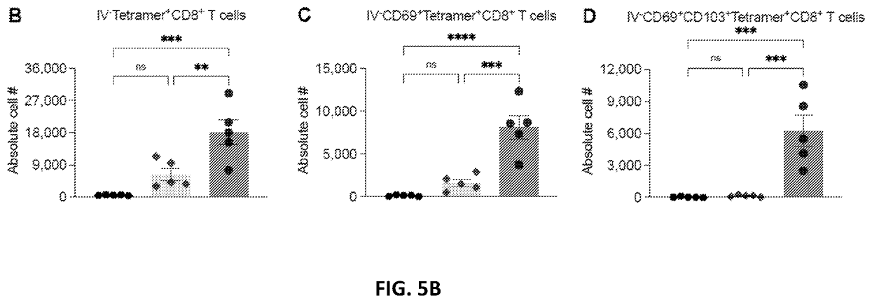

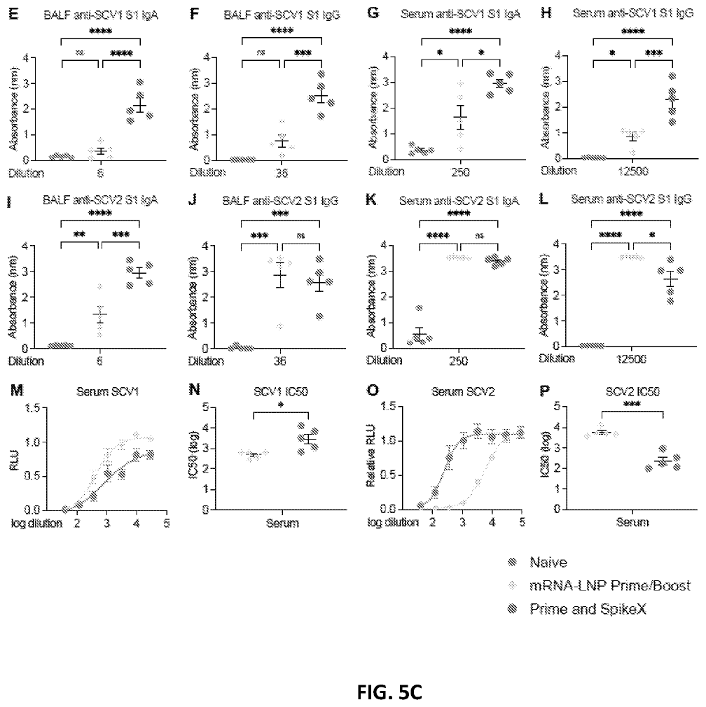

VeroE6 overexpressing hACE2 and TMPRSS2 (FIGS. 1A-1D) or Huh7.5 cell (FIGS. 5A-5C) were plated (3×10⁴) in each well of a 96-well plate the day before infection. On the day of infection, serum and BALF were heat-inactivated for 30 min at 56° C. FIGS. 1A-1D sera were tested at a starting dilution of 1:50 and BALF samples were tested at a starting dilution of 1:4, both with 8 twofold serial dilutions. FIGS. 5A-5C sera was tested at a starting dilution of 1:40 with 8 threefold serial dilutions. Serial dilutions mixed 1:1 with pseudoviruses and incubated for 1 hour at 37° C. Growth medium was then aspirated from the cells and replaced with 100 μL of serum/virus mixture. At 24 hours infection media was removed and plates flash frozen at −80° C. g passive lysis buffer (Promega) was added to each well and plates were incubated for 15 min at RT. 30 μg of Renilla-Glo Luciferase Assay System substrate (Promega) was added to each well and incubated at RT for 15 min. Luminescence was measured on a microplate reader (SpectraMax i3, Molecular Devices). IC50 was calculated as using Prism 9 (GraphPad Software) nonlinear regression.

### Example 12: IN Boosting with Unadjuvanted SARS-CoV-2 Spike Induces Mucosal Humoral Immunity

To assess the potential of IN unadjuvanted subunit vaccine boosting for the development of respiratory tract mucosal immunity, we decided to harness the strong systemic immunogenicity of mRNA-LNP. We additionally benefited from extensive SARS-CoV-2 spike engineering which helps stabilize the protein in its prefusion confirmation with the addition of a C-terminal T4 fibritin trimerization motif, six proline substitutions (F817P, A892P, A899P, A942P, K986P, V987P), and alanine substitutions (R683A and R685A) in the furin cleavage site. These sets of mutations have been shown to significantly enhance immunogenicity and increase protein stability, some of which are used in current vaccines.

We vaccinated K18-hACE2 mice with 1 μg of mRNA-LNP (Comirnaty) by IM injection (Prime), followed 14 days later by 1 μg of recombinant unadjuvanted spike protein by IN administration (Prime and Spike). Additional control groups include K18-hACE2 mice that received IM Prime only and mice that received IN spike only at boosting. Mice were euthanized at day 21 or 28 (7- or 14-days post boosting) and assessed for the development of mucosal humoral immunity (FIG. 1A).

First, we assessed anti-SARS-CoV-2 spike S1 IgG and IgA in nasal wash, bronchoalveolar lavage fluid (BALF), and serum. We found that only mice that received Prime and Spike developed high levels of anti-SARS-CoV-2 IgA and IgG in the nasal wash and BALF (FIG. 1B(B-E)). Neither IM Prime only nor IN spike only was sufficient for the development of mucosal antibodies. In the serum, IM Prime only was sufficient to induce low levels of IgA and IgG; however, Prime and Spike led to significant systemic boosting of both anti-spike S1 IgA and IgG (FIG. 1B(F,G)). These increases in antibody level correlated with increases in neutralization titers both in the BALF and serum (FIG. 1C(H-K)). These results indicate that single-dose unadjuvanted intranasal spike alone is not immunogenic, and that induction of a potent mucosal and systemic antibody response by unadjuvanted spike requires prior systemic priming, in this case by mRNA-LNP.

Tissue resident memory B cells (B_{RM}) in the lungs have been shown to assist in rapid recall response of antibody secreting B cells upon secondary heterologous challenge in mouse influenza models and may be an important local immune effector in protecting against SARSCoV-2. Using intravenous (IV) CD45 labeling combined with B cell tetramers specific for receptor binding domain (RBD) of the spike protein, we found that Prime and Spike leads to increased antigen specific B cells within lung tissue (IV⁻CD19⁺B220⁺Tetramer⁺) (FIG. 1D(L)). Given that the tetramer only assessed for RBD binding, we also looked at the polyclonal tissue response which likely represents a more complete set of B cells reactive to the entire spike within lung tissue. We found increases in class switched antibody secreting cells (ASC) (IVCD19^{+/-}CD138⁺) in lung tissue expressing IgA or IgG (FIG. 1D(M,N)), and we found increased class switched B_{RM} (IV⁻CD19⁺B220⁺IgD⁻IgM⁻CD38⁺) expressing IgA or IgG (FIG. 1D(O,P)). These results are consistent with increased mucosal antibody production and indicate that Prime and Spike elicits local B cell responses in the lung.

### Example 13: Prime and Spike Induces Mucosal T Cell Immunity

Given that we found that Prime and Spike induced mucosal humoral memory responses in the respiratory tract, we next wanted to assess the induction of lung tissue resident memory T cells (T_{RM}). While subunit vaccines have traditionally not been potent inducers of antigen specific T cell responses, we hypothesized that the immune memory generated by mRNA-LNP priming, which has been shown to be sufficient for induction of T cell memory responses in both animal models and humans, would enable subunit mediated T cell boosting responses. Similar to above, we combined CD45 IV labeling to differentiate circulating from immune cells within lung tissue with major histocompatibility complex (MHC) class I tetramer to a conserved sarbecovirus spike epitope (VNFNFNGL). We found significant induction of spike TV⁻ tetramer⁺ CD8⁺ T cells, which expressed canonical markers of T_{RM} including CD69⁺ and CD103⁺, within lung tissue (FIG. 2B(B-D)), the lower airway BALF (FIG. 2B(E-G)), and in the upper airway nasal turbinate (FIG. 2C(H-J)). Additionally, we found significant increases in antigen experienced CD4⁺ T cells (IVCD44⁻ CD4⁺), many of which also expressed markers of T_{RM} CD69⁺ and CD103⁺ both within lung tissue (FIG. 2C(K-M)) and from lower airway recovered from BALF (FIG. 2C(N-P)). These results indicate that Prime and Spike not only induces humoral mucosal responses, but also robustly elicits lung parenchyma and airway CD8⁺ T_{RM} and CD4⁺ T_{RM}.

### Example 14: Delayed Interval Prime and Spike is Sufficient to Induce Mucosal Immunity

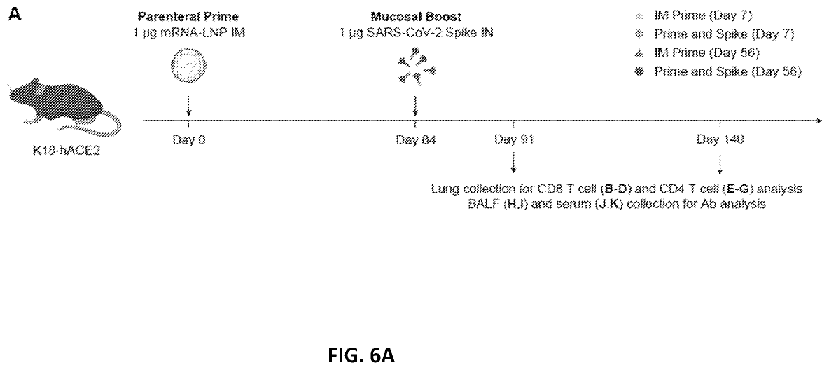

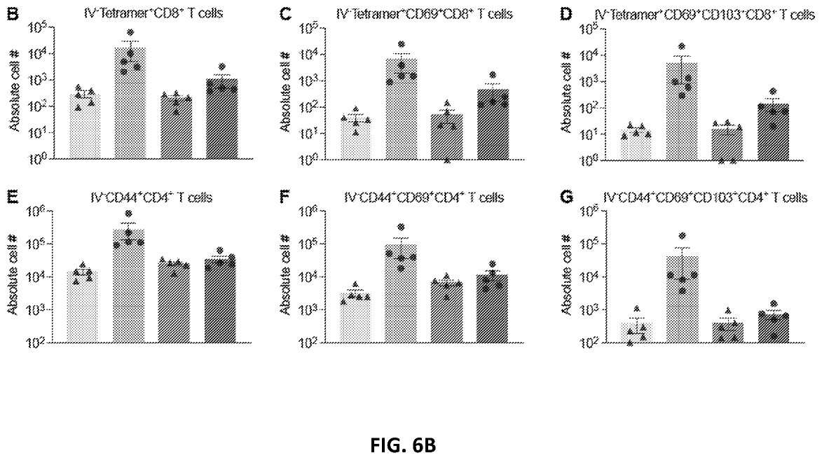

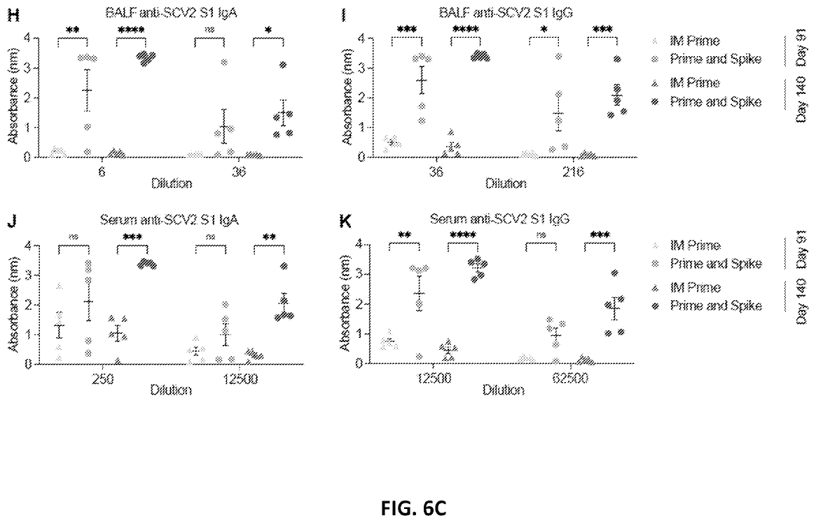

While we showed that Prime and Spike at a 14-day interval between priming and boosting resulted in significant induction of mucosal humoral and cellular immune memory responses, we wondered whether a delayed boost could also induce significant mucosal humoral and cellular responses. To test this question, K18-hACE2 mice that received 1 μg IM Prime were boosted with IN Spike 84 days later. We sampled humoral and cellular mucosal immune responses at day 91 (7 days post boost) and day 140 (56 days post boost) (FIG. 6A). We found that delayed IN Spike was sufficient to induce CD8⁺ T_{RM} which persisted for at least 56 days (FIG. 6B(B-D)). CD4⁺ T_{RM} were induced early at 7 days post boost; however, their longevity seemed to wane by 56 days, at least polyclonally (FIG. 6B(E-G)). Similar to the CD8⁺ T_{RM} response, we found not only adequate humoral response to delayed boosting, but strong and increasing mucosal IgA and IgG in BALF (FIG. 6C(H,I)), and strong and increasing serum IgA and IgG (FIG. 6C(J,K)) at 56 days post boosting. These results indicate that Prime and Spike given with a 3-month interval between doses is sufficient to elicit long lasting mucosal and systemic humoral and cellular immune responses.

### Example 15: IN Delivery of mRNA Polyplexes Also Mediates Mucosal Boosting

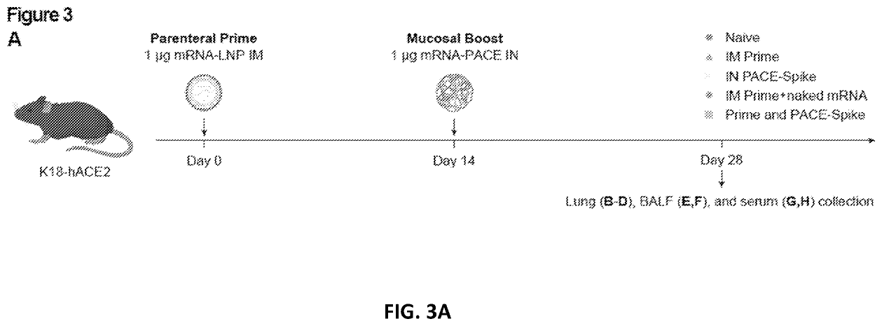

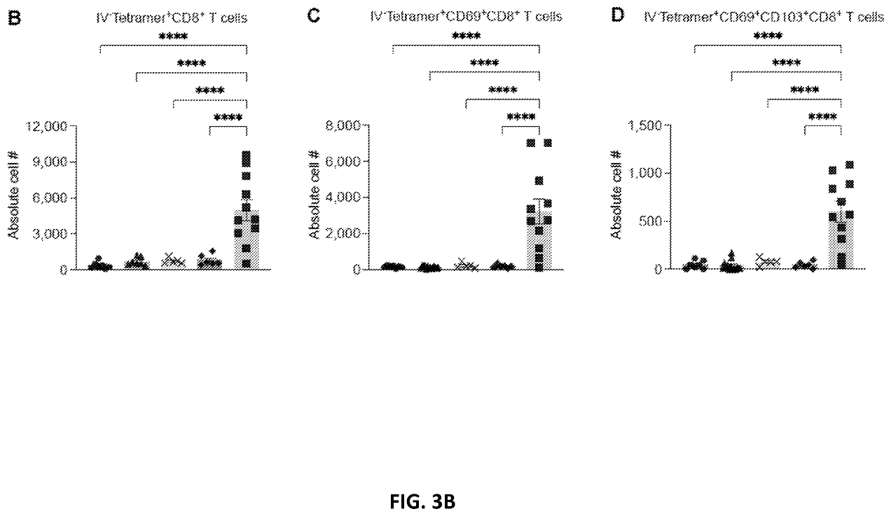

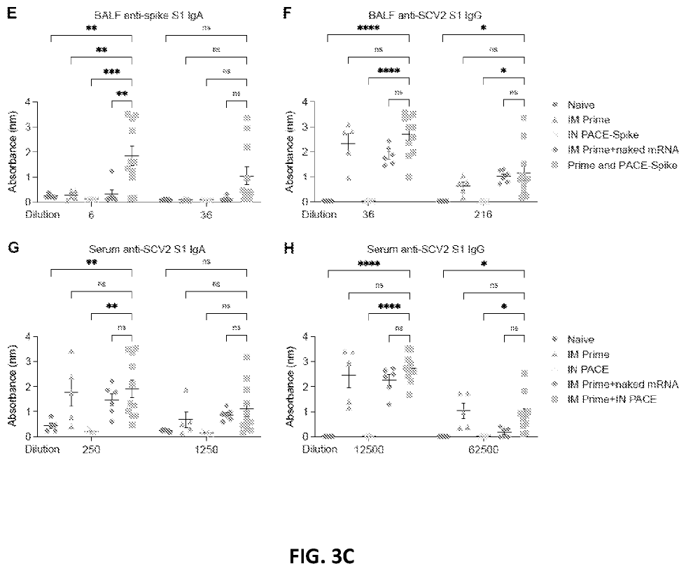

We next assessed the ability of alternative platforms for IN Spike boosting. Poly(amine-coester)s (PACE) are biodegradable terpolymers that have been developed to encapsulate and deliver nucleic acids such as mRNA or DNA to specified tissues in vivo depending on the properties of the polymer. Recent studies have shown that mRNA-LNP delivered to the respiratory tract is lethal in a dose dependent manner in mice. In contrast, PACE materials have been developed to be relatively immunologically silent, enabling administration to locations more susceptible to immunopathology such as the respiratory tract. To assess the safety and efficacy of PACE encapsulating mRNA encoding spike protein, mRNA was extracted from Comirnaty and encapsulated in PACE polyplexes. For vaccination, K18-hACE2 mice were injected with 1 μg IM Prime (mRNA-LNP), and 14 days later received 1 μg of mRNA encapsulated in PACE and administered IN (PACE-Spike). Additional control groups included PACE-Spike only and IM Prime+extracted mRNA without PACE encapsulation (naked mRNA) (FIG. 3A). Similar to what we found with Prime and Spike, Prime and PACE-Spike induced antigen specific CD8⁺ T_{RM} (IV-Tetramer⁺) expressing canonical tissue residency markers (CD69⁺ and CD103⁺) (FIG. 3B). Additionally, PACE-Spike boosted mice developed high levels of BALF anti-SARS-CoV-2 IgA; levels of BALF IgG and serum IgA and IgG were similar to IM Prime only mice (FIG. 3C). IM Prime followed by IN naked mRNA was unable to induce mucosal or systemic immune responses above that of IM Prime alone indicating that mRNA encapsulation by PACE was required for mucosal boosting. Additionally, a single dose of IN PACE-Spike alone was insufficient to elicit any detectable mucosal or systemic antibody response at this dose.

### Example 16: Prime and Spike or Prime and PACE-Spike in the Context of Waning mRNA-LNP Immunity Protects Against Lethal SARS-CoV-2 Challenge

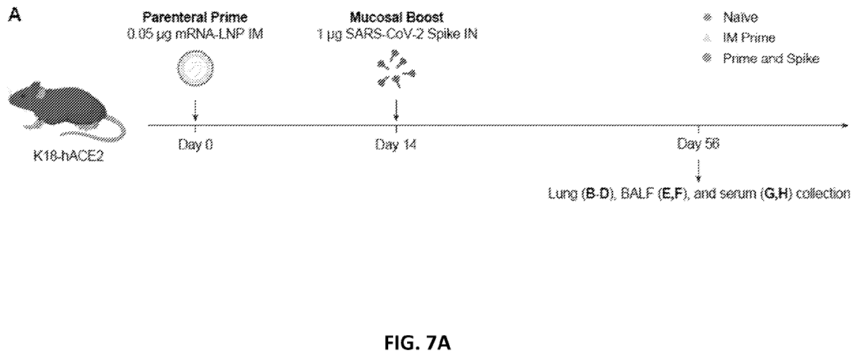

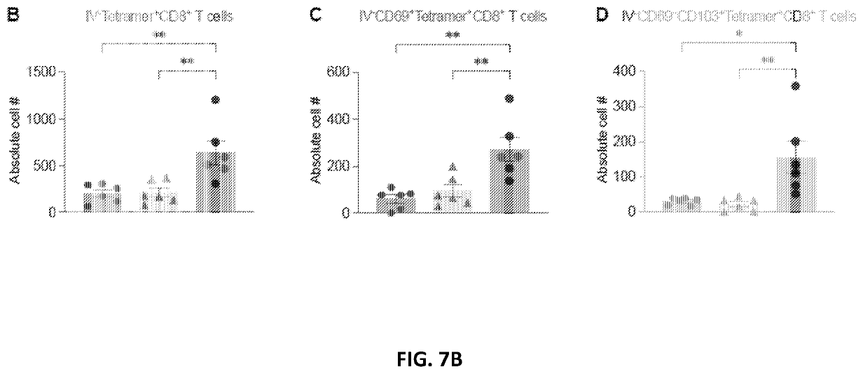

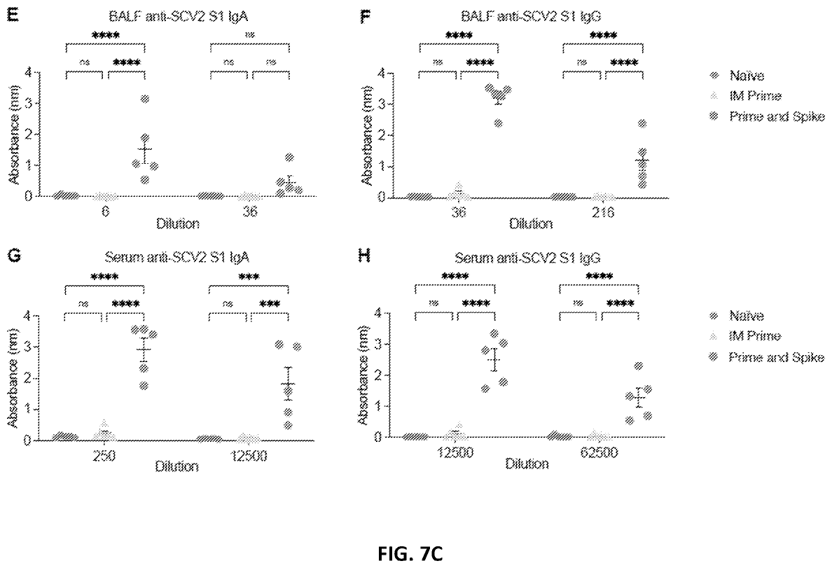

While current vaccines were initially extremely effective at eliciting protective immunity, waning antibody levels and immune evasion will necessitate boosters against SARS-CoV-2 for the foreseeable future; however, the best method for boosting remains a question. To test whether IN administration would provide an alternative protective boost, we utilized a low dose mRNALNP vaccine challenge model to mimic waning immunity; we performed single dose immunization with 0.05 μg mRNA-LNP. While these low dose mRNA-LNP vaccinated mice uniformly develop systemic antibody responses, we have previously shown that this dose is not sufficient to protect from SARS-CoV-2 challenge. Fourteen days post prime mice received IN Spike (1 μg unadjuvanted spike protein). Similar to 1 μg IM Prime mice that we described earlier, 0.05 μg IM primed mice boosted with IN Spike developed a significant increase in antigen specific CD8⁺ T_{RM} in the lungs as well as IgA and IgG in the BALF at 42 days post boost (FIG. 7A). These data also indicate that even very low levels of immune memory generated by low dose mRNA-LNP prime can be effectively boosted to induce mucosal and systemic humoral and cellular memory by unadjuvanted IN spike.

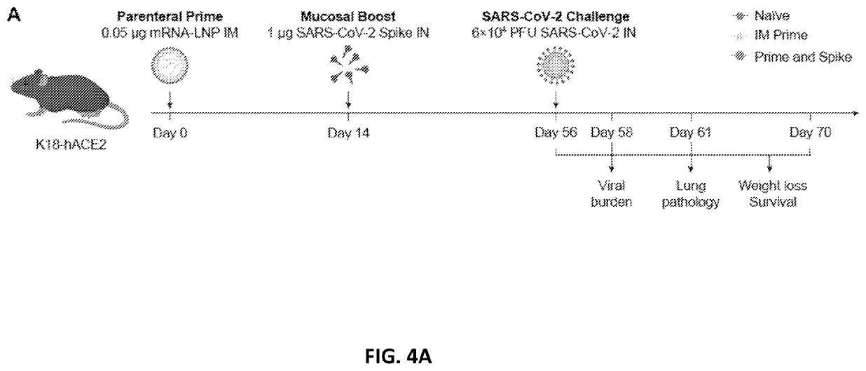

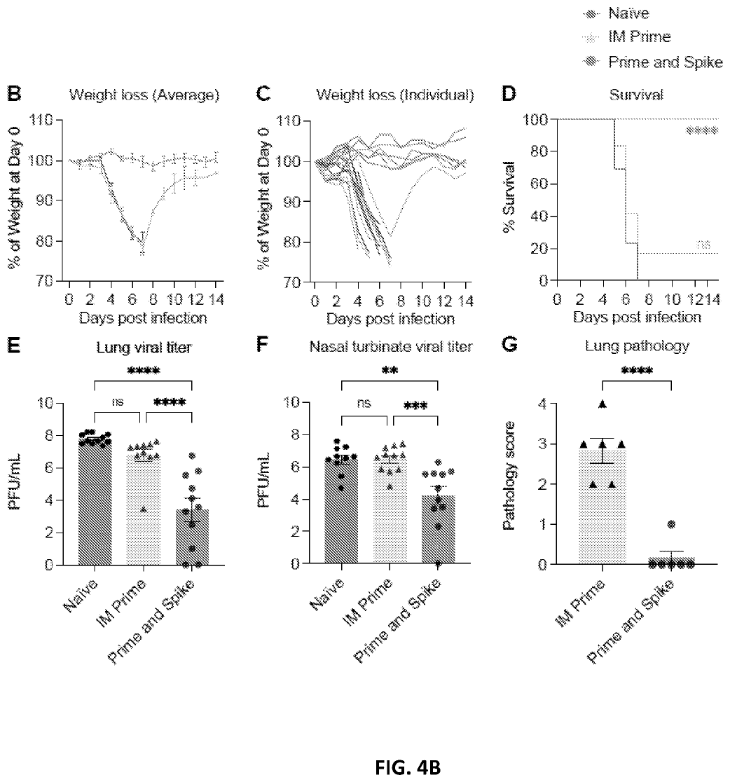

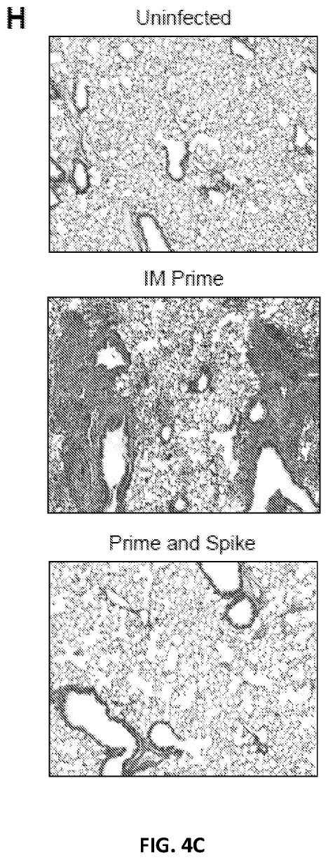

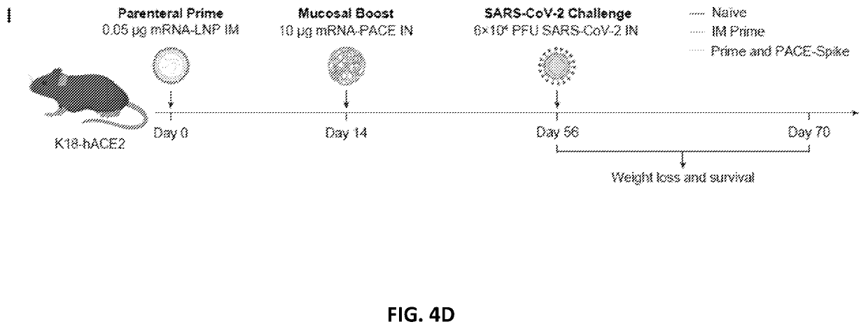

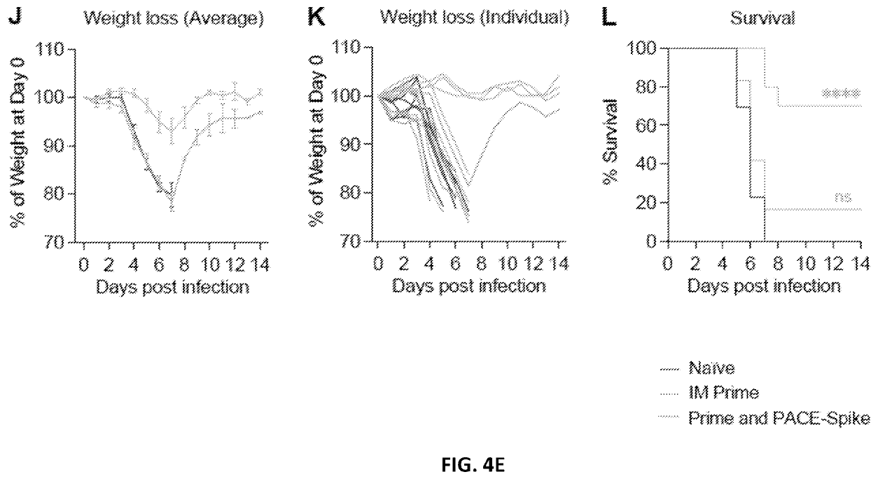

Naïve, low dose Prime only, and low dose Prime and Spike mice were then challenged with 6×10⁴ PFU homologous/ancestral WA1 strain SARS-CoV-2. Mice were either euthanized at 2 DPI and viral burden assessed by plaque assay from nasal turbinates and lungs, euthanized at 5 DPI and lungs assessed for pathology, or monitored for weight loss and mortality for 14 days (FIG. 4A). All mice given Prime and Spike were completely protected from weight loss or death, but neither naïve nor low dose Prime only mice were protected from viral challenge (FIG. 4B(B-D)). Additionally, this significant improvement in morbidity and mortality in mice receiving Prime and Spike was accompanied by reduced viral burden in both the upper respiratory tract (nasal turbinates) and lower respiratory tract (lungs) (FIG. 4B(E,F)). Further, Prime and Spike led to significant protection from lung pathology with only 1 of 6 mice developing limited mononuclear infiltrates at 5 DPI, while the remaining mice were completely protected with lung architecture similar to that seen in uninfected mice (FIG. 4B(G), FIG. 4C). To assess the protective capacity of a mRNA-PACE IN boost, we again made use of the low-dose prime mRNA-LNP mice, and boosted with 10 μg of mRNA-PACE IN. We found that Prime and PACE-Spike resulted in significant protection from morbidity and mortality (FIG. 4D, FIG. 4E). These data suggest that either IN unadjuvanted spike or mRNA-PACE encoding spike sufficiently boost mucosal immunity to protect from COVID-19 like pulmonary disease and mortality in a preclinical mouse model. These results also highlight the robustness, versatility, and safety of this vaccine strategy as intranasal boosting of systemic mRNA-LNP priming by either modality is sufficient to induce mucosal immunity and to provide protection against lethal SARS-CoV-2 challenge.

### Example 17: Prime and Spike Elicits Robust Systemic Immunity Similar to Parenteral mRNA-LNP Based Boost

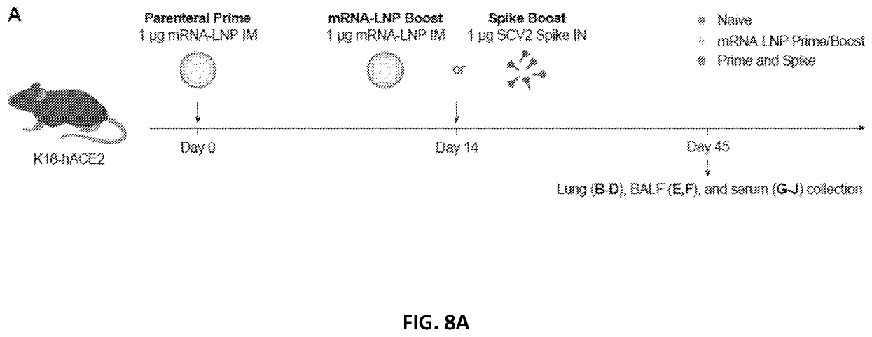

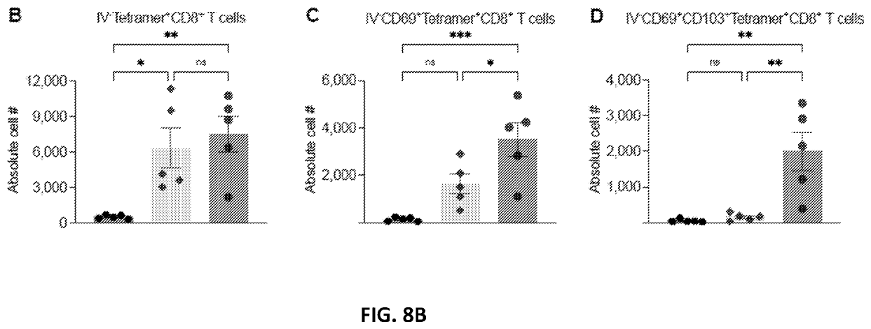

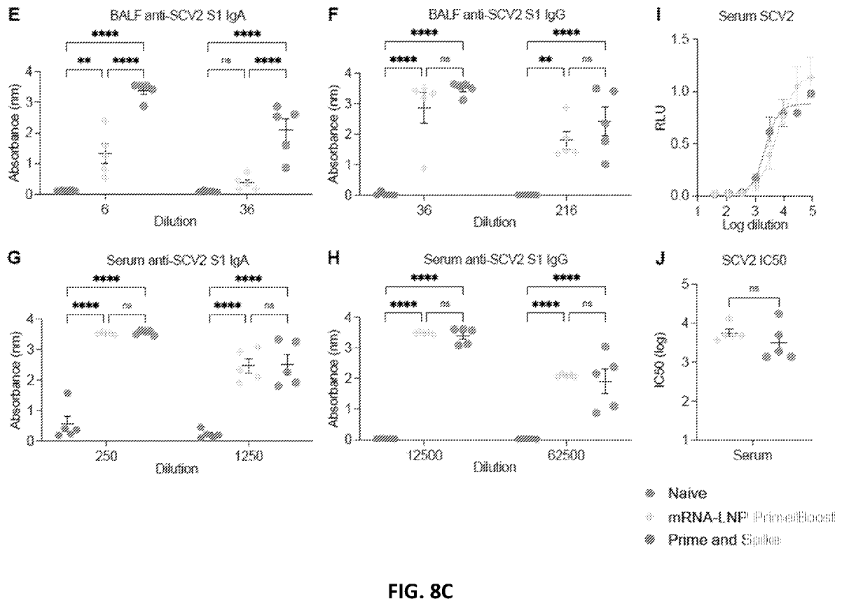

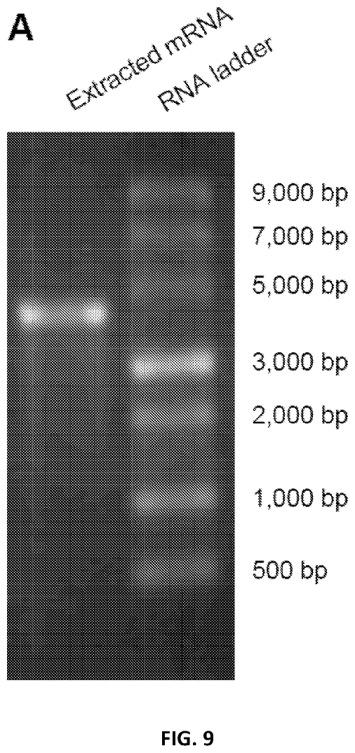

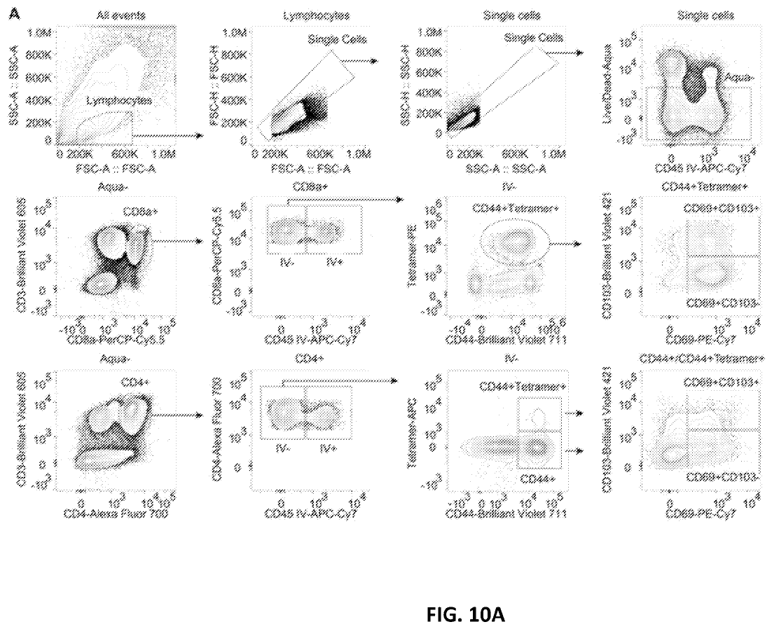

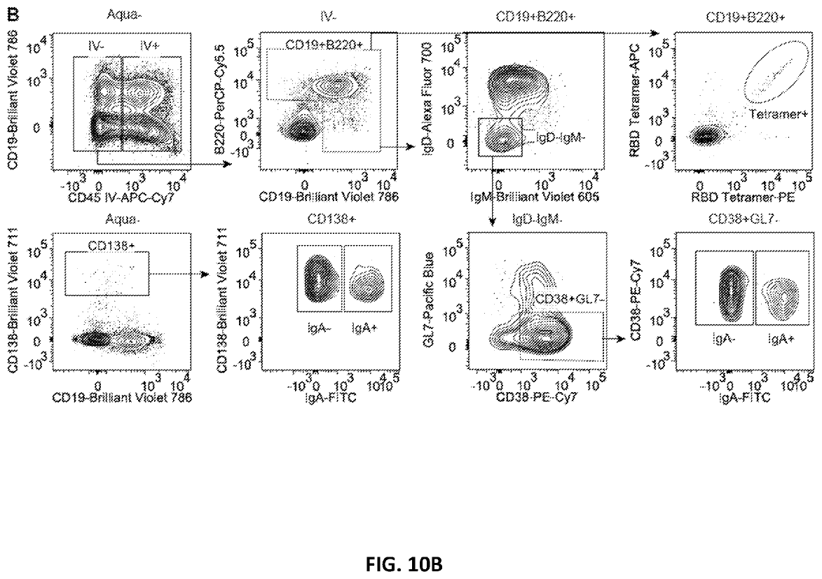

IM injected mRNA-LNP based vaccines are the current standard recommended boosting strategy in many countries as immunogenicity and vaccine efficacy studies have most concentrated on this method of boosting. To compare Prime and Spike to IM mRNA-LNP prime/boost, we primed K18-hACE2 mice with 1 μg of mRNA-LNP, followed 14 days later by either 1 μg IN Spike or 1 μg IM mRNA-LNP. Mice were euthanized 31 days post boost and antigen specific CD8⁺ T_{RM} were assessed by flow cytometry, antibodies from BALF and serum were assessed by ELISA, and VSV pseudovirus neutralization assay was performed to assess serum antibody neutralization response (FIG. 8A-FIG. 8C). We found that both IM mRNA-LNP boosted and IN Spike boosted animals developed increased levels of extravascular (IV⁻) Tetramer⁺CD8⁺ T cells; however, only IN spike boosted animals developed CD8⁺ T_{RM} that express CD69⁺ and CD103⁺ in the lung (FIG. 8B). By ELISA we found that only IN spike boosted animals developed anti-SARS-CoV-2 IgA in the BALF. BALF IgG levels were similar in IM mRNA-LNP and IN spike boosted mice, possibly representing transcytosis from elevated systemic antibody levels in the IM mRNA-LNP boosted mice. We similarly found equivalent serum levels of anti-SARS-CoV-2 IgA and IgG in IM mRNA-LNP and IN spike boosted mice. Neutralization assays from serum also showed similar IC50 between IM mRNA-LNP and IN Spike boosting. These data demonstrate that Prime and Spike induces similar systemic neutralizing antibody levels to IM mRNA-LNP boosting, which has been shown to be a correlate of protection, and uniquely elicits mucosal IgA and CD8⁺ T_{RM}.

### Example 18: Heterologous Spike Robustly Elicits Cross-Reactive Immunity without Original Antigenic Sin

The experiments above clearly demonstrate that boosting at a distinct anatomic location, in this case the respiratory mucosa, either by unadjuvanted subunit spike or by PACE-Spike encoding spike, enables the formation of new mucosal immune memory at the newly boosted site and enhances systemic immunity to that antigen. In both unadjuvanted subunit spike and mRNAPACE, the boosting antigen is homologous to the systemic priming antigen (mRNA-LNP). Current circulating strains of SARS-CoV-2, notably Delta and Omicron, have significant changes to the spike protein sequence and structure. Delta harbors T19R, G142D, Δ156-157, R158G, Δ213-214, L452R, T478K, D614G, P681R, and D950N mutations, whereas Omicron harbors A67V, Δ69-70, T95I, G142D, Δ143-145, N211I, L212V, ins213-214RE, V215P, R216E, G339D, S371L, S373P, S375F, K417N, N440K, G446S, S477N, T478K, E484A, Q493R, G496S, Q498R, N501Y, Y505H, T547K, D614G, H655Y, N679K, P681H, N764K, D796Y, N856K, Q954H, N969K, and L981F mutations. These mutations have made both Delta and Omicron transmit more rapidly and evade pre-existing humoral immunity, and it is likely that future variants will diverge even more, suggesting a boosting strategy that elicits broadly reactive immunity will be necessary to neutralize future variants. To test the ability of an unadjuvanted heterologous spike protein for IN boosting, we primed K18-hACE2 mice with 1 μg mRNA-LNP followed 14 days later by boosting with 5 μg of SARS-CoV-1 spike containing trimer stabilizing mutations (R667A, K968P, V969P), or Prime and SpikeX (FIG. 5A). While SARS-CoV-1 is a related sarbecovirus, its spike protein only shares 76% homology with the original SARS-CoV-2 spike sequence that is encoded by currently used mRNA-LNP vaccines. For comparison we also boosted mRNA-LNP primed mice with 1 μg IM mRNA-LNP. At 31 days post boost, using CD45 IV labeling, we found significantly increased TV⁻ Tetramer⁺ CD8⁺ T cells that express canonical T_{RM} markers CD69⁺ and CD103⁺ (FIG. 5B). As noted earlier, this MHC I tetramer sequence is highly conserved within the sarbecovirus family, which both SARS-CoV-1 and SARS-CoV-2 are a part of. Next, we assessed the development of anti-SARS-CoV-1 antibodies in BALF and serum and found significant increases of anti-SARS-CoV-1 IgA and IgG in both the respiratory mucosa and the circulation in Prime and SpikeX relative to IM mRNA-LNP prime/boost. Consistent with previous studies, we find that two doses of SARS-CoV-2 mRNALNP is sufficient to induce detectable antibodies that bind SARS-CoV-1 spike. Next, we assessed anti-SARS-CoV-2 antibodies in BALF and serum. We found that Prime and SpikeX induced higher BALF IgA than IM SARS-CoV-2 mRNA-LNP prime/boost. We found similar levels of anti-SARS-CoV-2 IgG in BALF which likely represents the increased serum IgG that we found (FIG. 5C(I-L)). Next, using VSV-based pseudovirus neutralization assay we show that serum from Prime and SpikeX mice develops higher neutralization titers against SARS-CoV-1 than mice boosted with I SARS-CoV-2 mRNA-LNP (FIG. 5C(M,N)). Similarly, and consistent with serum IgG levels, IM SARS-CoV-2 mRNA-LNP prime/boost mice have significantly higher neutralization titers against SARS-CoV-2 than Prime and SpikeX mice (FIG. 5C(O,P)). Taken together, these data indicate that IN boosting with unadjuvanted heterologous spike protein can induce potent mucosal cellular and humoral memory against significantly divergent spike protein in the absence of original antigenic sin.

### Example 19: Prime and Spike Induces Mucosal Immunity Against SARS-CoV-2

During the past two years of SARS-CoV-2 pandemic, vaccines containing modified mRNA encapsulated in lipid nanoparticles (LNP) have been very effective. Phase 3 clinical trials and subsequent post marketing vaccine effectiveness studies initially showed >90% vaccine efficacy against symptomatic disease. Unfortunately, more recent studies have demonstrated decreasing vaccine effectiveness in terms of asymptomatic infection as well as symptomatic and severe infections starting around 4 months post second dose with mRNA-LNP based regimens. This study is to analyze Prime and Spike's effect on the patients who previously were vaccinated against or infected by SARS-CoV-2. To this end, the patients who previously were vaccinated against or infected by SARS-CoV-2 are divided evenly in two groups. One group is a control group and is administered a intranasal formulation containing a placebo; another group is administered a intranasal formulation containing the mRNA vaccines approved by FDA. The administration are given at 1 month, two months, three months, four months, five months, six months, seven months, eight months, nine months, ten months, eleven months, or twelve months after the infection or vaccination of the patients. To evaluate for effectiveness of Prime and Spike, CD8⁺ T cells, CD4⁺ T cells, memory T cells (T_{RM}) and B cells (B_{RM}) as well as mucosal IgG and dimeric IgA will be measured at the mucosal site where the vaccine is administered.

### Example 20: Prime and Spike Induces Mucosal Immunity Against Human Papillomavirus (HPV)

This study is to analyze Prime and Spike's effect on the patients who previously were vaccinated against or infected by HPV. To this end, the patients who previously were vaccinated against or infected by HPV are divided evenly in two groups. One group is a control group and is administered a vaginal formulation containing a placebo; another group is administered a vaginal formulation containing the vaccine against HPV. The administration are given at 1 month, two months, three months, four months, five months, six months, seven months, eight months, nine months, ten months, eleven months, or twelve months after the infection or vaccination of the patients. To evaluate for effectiveness of Prime and Spike, CD8⁻ T cells, CD4⁺ T cells, memory T cells (T_{RM}) and B cells (B_{RM}) as well as mucosal IgG and dimeric IgA will be measured at the mucosal site where the vaccine is administered.

### Example 21: Prime and Spike Induces Mucosal Immunity Against Rotavirus

This study is to analyze Prime and Spike's effect on the patients who previously were vaccinated against or infected by rotavirus. To this end, the patients who previously were vaccinated against or infected by rotavirus are divided evenly in two groups. One group is a control group and is administered an oral formulation containing a placebo; another group is administered an oral formulation containing the vaccine against rotavirus. The administration are given at 1 month, two months, three months, four months, five months, six months, seven months, eight months, nine months, ten months, eleven months, or twelve months after the infection or vaccination of the patients. To evaluate for effectiveness of Prime and Spike, CD8⁺ T cells, CD4⁺ T cells, memory T cells (T_{RM}) and B cells (B_{RM}) as well as mucosal IgG and dimeric IgA will be measured at the mucosal site where the vaccine is administered.

1. A method of enhancing an immune response to an antigen in a human in need thereof, the method comprising administering to the subject an effective amount of a pharmaceutical composition comprising the antigen or a nucleic acid encoding the antigen at a mucosal site, wherein the human has been previously vaccinated against or infected by a virus.

2. The method of claim 1, wherein the human has been parenterally vaccinated against the virus.

3. The method of claim 1, wherein the antigen is a multivalent antigen.

4. A method of enhancing an immune response to an antigen in a human in need thereof, the method comprising administering to the subject an effective amount of a pharmaceutical composition comprising the antigen at a mucosal site, wherein the human has elevated antibodies, memory B cells, effector CD4⁻ and/or CD8⁺ T cells.

5. The method of claim 4, wherein the elevated antibodies, memory B cells, effector CD4⁺ and/or CD8⁺ T cells are caused by a previous vaccination against a virus.

6. The method of claim 4, wherein the elevated antibodies, memory B cells and effector CD4⁺ and CD8⁺ T cells are caused by a previous infection of a virus.

7. The method of claim 5 or claim 6, wherein the elevated antibodies are immunoglobulin G (IgG), IgM, IgA, or combinations thereof.

8. The method of any one of claims 1-7, wherein the mucosal site is nasal.

9. The method of any one of claims 1-8, wherein the antigen comprises at least a protein or polypeptide.

10. The method of claim 1, wherein the nucleic acid is DNA or RNA.

11. The method of claim 1, wherein the nucleic acid is mRNA.

12. The method of claim 11, wherein the mRNA is N1-methyl-pseudouridine-modified mRNA.

13. The method of claim 11, wherein the mRNA is pseudouridine-modified mRNA.

14. The method of any one of claims 1-13, wherein the antigen is derived from a microbial pathogen.

15. The method of claim 14, wherein the microbial pathogen is a mycobacterium, bacterium, fungus, virus, parasite, or prion.

16. The method of any one of claims 1-15, wherein the virus is selected from the group consisting of rotavirus, norovirus, adenovirus, astrovirus, variants thereof, and any combination thereof.

17. The method of any one of claims 1-15, wherein the virus is selected from the group consisting of influenza virus, respiratory syncytial virus, parainfluenza viruses, metapneumovirus, rhinovirus, coronavirus, adenovirus, bocavirus, variants thereof, and any combination thereof.

18. The method of any one of claims 1-15, wherein the virus is selected from the group consisting of herpes simplex virus type 1 (HSV-1), herpes simplex virus type 2 (HSV-2), human papillomavirus (HPV), variants thereof, and any combination thereof.

19. The method of any one of claims 1-15, wherein the virus is selected from the group consisting of human immunodeficiency virus (HIV), hepatitis A, hepatitis B, hepatitis C, herpes virus, adenovirus, poliomyelitis, Japanese encephalitis, smallpox, influenza virus, flaviviruses, echovirus, rhinovirus, coxsackie virus, coronavirus, respiratory syncytial virus (RSV), mumps virus, rotavirus, measles virus, rubella virus, parvovirus, vaccinia virus, human T-lymphotropic virus (HTLV), dengue virus, human papillomavirus (HPV), molluscum virus, poliovirus, rabies virus, JC virus, arboviral encephalitis virus, SARS-CoV-2, Henoch-Schonlein purpura (HSP), an RNA virus, a DNA virus, variants thereof, and any combination thereof.

20. The method of claim 19, wherein the RNA virus selected from the group consisting of common cold, influenza, SARS, MERS, Covid-19, Dengue Virus, hepatitis C, hepatitis E, West Nile fever, Ebola virus disease, rabies, polio, mumps, measles, variants thereof, and any combination thereof.

21. The method of claim 19, wherein the DNA virus selected from the group consisting of herpes simplex virus, cytomegalo virus, varicella zoster virus, Epstein-Barr virus, roseolo virus, human herpesvirus-7, Kaposi's sarcoma-associated virus, variants thereof, and any combination thereof.

22. The method of any of claims 1-21, wherein the antigen is administered by mucosal delivery.

23. The method of any one of claims 1-22, wherein the antigen is administered to a mucosal tissue of the human subject.

24. The method of claim 23, the mucosal tissue is selected from the group consisting of anterior nostril, nasal sinus, rectal, vaginal, esophagus, urethral, sublingual and buccal.

25. The method of any one of claims 1-24, wherein the pharmaceutical composition is administered intranasally.

26. The method of any one of claims 1-24, wherein the pharmaceutical composition does not comprise an adjuvant.

27. The method of any one of claims 1-24, wherein the pharmaceutical composition comprises an adjuvant.

28. The method of any one of claims 1-27, wherein the pharmaceutical composition comprises a lipid nanoparticle (LNP).

29. The method of claim 28, wherein the antigen is encapsulated within the lipid nanoparticle (LNP).

30. The method of claim 28, wherein the lipid nanoparticle (LNP) comprises at least one cationic lipid.

31. The method of claim 30, wherein the at least one cationic lipid comprises 1,2-dimyristoyl-sn-glycero-3-ethylphosphocholine (DMEPC), 1,2-di-O-octadecenyl-3-trimethylammonium propane (DOTMA) and/or 1,2-dioleoyl-3-trimethylammonium propane (DOTAP).

32. The method of claim 28, wherein the lipid nanoparticle (LNP) comprises poly(amine-co-ester) (PACE) polymer.

33. The method of claim 28, wherein the lipid nanoparticle (LNP) further comprise at least one phospholipid.

34. The method of claim 33, wherein the at least phospholipid comprises 1,2-distearoyl-sn-glycero-3-phosphocholine (DSPC), 1,2-Dioleoyl-sn-glycero-3-phosphoethanolamine (DOPE), cholesterol (Chol), 1-palmitoyl-2-oleoyl-sn-glycero-3-phosphocholin (POPC) and/or 1,2-dioleoyl-sn-glycero-3-phosphocholine (DOPC).

35. The method of any one of claims 1-34, wherein the human has been vaccinated against or infected by the virus about one week ago, two weeks ago, three weeks ago, one month ago, two months ago, three months ago, four months ago, five months ago, six months ago, seven months ago, eight months ago, nine months ago, ten months ago, eleven months ago, or twelve months ago.

36. A method of enhancing an immune response to SARS-CoV-2 in a human in need thereof, the method comprising administering to the subject an effective amount of a pharmaceutical composition comprising at least one mRNA at a mucosal site, wherein the human has been previously vaccinated against or infected by a virus.

37. The method of claim 36, wherein the human has been previously vaccinated with one or more COVID-19 vaccines selected from the group consisting of BNT162b2 (Pfizer/BioNTech), mRNA-1273 (Moderna), AZD1222/ChAdOxl (AstraZeneca/Oxford Univ), Ad5-vectored COVID-19 vaccine (CanSino Biologies), CoronaVac (Sinovac), NVX-CoV2373 (Novavax), and combinations thereof.

38. The method of claim 36, wherein the at least one mRNA encodes the spike protein of SARS-CoV-2 or variants thereof or a fragment thereof.

39. The method of claim 36, wherein the pharmaceutical composition comprises mRNAs encoding two or more different antigens.

40. The method of claim 36, wherein the two or more antigens are spike proteins of SARS-CoV-2 or variants thereof or a fragment thereof.

41. The method of claim 40, wherein the two or more antigens comprise at least one mutation listed in Table 1.

42. The method of any one of claims 36-41, wherein the pharmaceutical composition does not comprise an adjuvant.

43. The method of any one of claims 36-41, wherein the pharmaceutical composition comprises an adjuvant.

44. The method of any one of claims 36-43, wherein the pharmaceutical composition further comprises a lipid nanoparticle (LNP).

45. The method of claim 44, wherein the at least one mRNA is encapsulated within the lipid nanoparticle (LNP).

46. The method of claim 45, wherein the lipid nanoparticle (LNP) comprises at least one cationic lipid.

47. The method of claim 46, wherein the at least one cationic lipid comprises 1,2-dimyristoyl-sn-glycero-3-ethylphosphocholine (DMEPC), 1,2-di-O-octadecenyl-3-trimethylammonium propane (DOTMA) and/or 1,2-dioleoyl-3-trimethylammonium propane (DOTAP).

48. The method of claim 45, wherein the lipid nanoparticle (LNP) comprises poly(amine-co-ester) (PACE) polymer.

49. The method of claim 45, wherein the lipid nanoparticle (LNP) further comprise at least one phospholipid.

50. The method of claim 49, wherein the at least phospholipid comprises 1,2-distearoyl-sn-glycero-3-phosphocholine (DSPC), 1,2-Dioleoyl-sn-glycero-3-phosphoethanolamine (DOPE), cholesterol (Chol), 1-palmitoyl-2-oleoyl-sn-glycero-3-phosphocholin (POPC) and/or 1,2-dioleoyl-sn-glycero-3-phosphocholine (DOPC).

51. The method of any one of claims 36-50, wherein the lipid nanoparticle has an average diameter in the range of from about 50 nm to about 1000 nm.

52. The method of any one of claims 36-50, wherein the lipid nanoparticle has an average diameter in the range of from about 50 nm to about 400 nm, from about 50 nm to about 200 nm, from about 200 nm to about 1000 nm, from about 200 nm to about 800 nm, or from about 300 nm to about 600 nm.

53. The method of any one of claims 1-52, wherein the immune response is a mucosal immune response.

54. The method of claim 53, wherein the mucosal immune response is an antigen-specific IgA antibody production.

55. The method of claim 53, wherein the mucosal immune response is an antigen-specific IgG antibody production.

56. The method of claim 53, wherein the mucosal immune response is an antigen-specific IgM antibody production.

57. The method of claim 36, wherein the human has elevated neutralizing antibody levels caused by a previous vaccination against the virus selected from the group consisting of MERS-CoV, SARS-CoV-1, SARS-Cov-2, and variants thereof.

58. The method of claim 36, wherein the human has elevated neutralizing antibody levels caused by a previous infection of the virus selected from the group consisting of MERS-CoV, SARS-CoV-1, SARS-Cov-2, and variants thereof.

59. The method of any of claim 57 or claim 58, wherein the elevated neutralizing antibody is IgG, IgM, IgA, or combinations thereof.

60. The method of claim 59, wherein the human has elevated IgG antibody caused by a previous vaccination against the virus selected from the group consisting of MERS-CoV, SARS-CoV-1, SARS-Cov-2, and variants thereof.

61. The method of claim 59, wherein the human has elevated IgM antibody caused by a previous vaccination against the virus selected from the group consisting of MERS-CoV, SARS-CoV-1, SARS-Cov-2, and variants thereof.

62. The method of claim 59, wherein the human has elevated IgA antibody caused by a previous vaccination against the virus selected from the group consisting of MERS-CoV, SARS-CoV-1, SARS-Cov-2, and variants thereof.

63. The method of claim 59, wherein the human has elevated IgG antibody caused by a previous infection of the virus selected from the group consisting of MERS-CoV, SARS-CoV-1, SARS-Cov-2, and variants thereof.

64. The method of claim 59, wherein the human has elevated IgM antibody caused by a previous infection of the virus selected from the group consisting of MERS-CoV, SARS-CoV-1, SARS-Cov-2, and variants thereof.

65. The method of claim 59, wherein the human has elevated IgA antibody caused by a previous infection of the virus selected from the group consisting of MERS-CoV, SARS-CoV-1, SARS-Cov-2, and variants thereof.

66. The method of any one of claims 36-65, wherein the human has been vaccinated against or infected by the virus about one week ago, two weeks ago, three weeks ago, one month ago, two months ago, three months ago, four months ago, five months ago, six months ago, seven months ago, eight months ago, nine months ago, ten months ago, eleven months ago, or twelve months ago.

67. The method of any one of claims 35-65, wherein the SARS-CoV-2 variant is selected from the group consisting of Alpha (B.1.1.7 and Q lineages), Beta (B.1.351 and descendent lineages), Gamma (P.1 and descendent lineages), Delta (B.1.617.2 and AY lineages), Epsilon (B.1.427 and B.1.429), Eta (B.1.525), Iota (B.1.526), Kappa (B.1.617.1), 1.617.3, Mu (B.1.621, B.1.621.1), Zeta (P.2), Mu (B.1.621, B.1.621.1), Omicron (Pango lineages B.1.1.529, BA.1, BA.1.1, BA.2, BA.3), and combinations thereof.

68. The method of any one of claims 36-67, wherein the at least one mRNA is N1-methyl-pseudouridine-modified mRNA.

69. The method of any one of claims 36-67, wherein the at least one mRNA is pseudouridine-modified mRNA.


SEQUENCE LISTING (2 sequences)

SEQ ID NO: 1 - DNA, 3822 bp, Coronavirus
  misc_feature 1..3822: note = coronavirus 2 isolate Wuhan-Hu-1
  source 1..3822: mol_type = genomic DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 2 - protein, 1273 aa, Coronavirus
  REGION 1..1273: note = MISC_FEATURE - coronavirus 2 isolate Wuhan-Hu-1
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT